(19) United States
(12) Patent Application Publication
(10) Pub. No.: US 2021/0275103 A1
(43) Pub. Date: Sep. 9, 2021

(54) BIOLOGICAL INFORMATION PROCESSING APPARATUS AND INFORMATION PROCESSING METHOD

(71) Applicant: SONY CORPORATION, TOKYO (JP)

(72) Inventor: TAKANORI ISHIKAWA, TOKYO (JP)

(21) Appl. No.: 17/250,331

(22) PCT Filed: May 28, 2019
(86) PCT No.: PCT/JP2019/020979

§ 371 Date: Jan. 6, 2021

(30) Foreign Application Priority Data
Jul. 17, 2018 (JP) 2018-134456

(51) Int. Cl. A61B 5/00; A61B 5/11

(52) U.S. Cl. CPC A61B 5/721; A61B 5/4266; A61B 5/725; A61B 2562/0219; A61B 5/6843; A61B 5/1118; A61B 2562/0247; A61B 5/7221

(57) ABSTRACT

It is possible to accurately reduce body motion noise included in an observation signal of biological information. Provided is a biological information processing apparatus including a noise reduction processing unit configured to calculate an error signal obtained by subtracting body motion noise included in an observation signal from a first sensor unit configured to measure biological body affect as an observation signal, on the basis of a body motion signal from a second sensor unit configured to measure a body motion change, and/or a pressure signal from a third sensor unit configured to measure a pressing force change in skin.

### TECHNICAL FIELD

The present disclosure relates to a biological information processing apparatus and an information processing method.

### BACKGROUND ART

In recent years, various measurement technologies for determining biological information have been considered.

For example, in Patent Document 1, a Galvanic Skin Response (GSR) is used as one of biological information. Electrical activities of the skin of a user that can also be used as biological information like a GSR will also be collectively referred to as an Electro-Dermal Activity (EDA). The EDA will also be referred to as an Electro-Dermal Response (EDR). Furthermore, a Skin Potential Activity (SPA) is also included in an EDA. The EDA is not limited to the example of Patent Document 1 and widely used as a method for detecting an activity of an autonomic nervous system of a user, for example.

Patent Document 2 discloses an analysis apparatus including a data acquisition unit that acquires data of an impedance or a conductance measured by flowing an alternating current between an electrode pair being in contact with the skin of the user, and an analysis unit that extracts biological information of the user from the data. Patent Document 2 discloses that measurement accuracy can be improved while minimizing restriction on the measurement of a skin impedance or skin conductance.

### CITATION LIST

### Patent Document

Patent Document 1: Japanese Patent Application Laid-Open No. 2009-39157
Patent Document 2: Japanese Patent Application Laid-Open No. 2016-97159

### SUMMARY OF THE INVENTION

### Problems to be Solved by the Invention

In a case where biological information in daily life is measured by a sensor as an observation signal, the observation signal sometimes includes body motion noise.

In view of the foregoing, the main object of the present technology is to provide a biological information processing apparatus and a biological information processing method that can accurately reduce body motion noise included in an observation signal of biological information.

### Solutions to Problems

The present technology provides a biological information processing apparatus including a noise reduction processing unit configured to calculate an error signal obtained by subtracting body motion noise included in an observation signal from a first sensor unit configured to measure biological body affect as an observation signal,

on the basis of a body motion signal from a second sensor unit configured to measure a body motion change, and/or a pressure signal from a third sensor unit configured to measure a pressing force change in skin.

According to an aspect of the present technology, the first sensor may be a sweat sensor unit.

According to an aspect of the present technology, the noise reduction processing unit may be configured to regard either the body motion signal or the pressure signal as a reference signal, and calculate an error signal by subtracting body motion noise from the observation signal, using the reference signal.

According to an aspect of the present technology, an active state analysis unit configured to analyze an active state on the basis of the observation signal, the body motion signal and/or the pressure signal, and determine a reference signal from the body motion signal or the pressure signal on the basis of the analysis result may be further included.

According to an aspect of the present technology, a bandpass filter unit configured to extract a fluctuation component from the signal using a bandpass filter may be further included.

According to an aspect of the present technology, an output signal quality calculation unit configured to determine a reduction state of body motion noise on the basis of a relationship between observation signal power calculated from the observation signal and error signal power calculated from the error signal may be further included.

According to an aspect of the present technology, a postprocessing filter unit configured to further reduce residual noise included in the error signal, by low pass filter processing may be further included.

According to an aspect of the present technology, the active state analysis unit may further include a second sensor analysis unit configured to determine an active state, and the active state analysis unit may be configured to output, in a case where it is determined in the second sensor analysis unit that the body motion signal is equal to or larger than a threshold value, the body motion signal to the noise reduction processing unit as a reference signal.

According to an aspect of the present technology, the active state analysis unit may further include a third sensor analysis unit configured to determine a quasi-rest state, and the active state analysis unit may be configured to output, in a case where it is determined in the third sensor analysis unit that the pressing force signal is equal to or larger than a threshold value, the pressing force signal to the noise reduction processing unit as a reference signal.

According to an aspect of the present technology, the active state analysis unit may be configured to output, in a case where it is determined in the third sensor analysis unit that the pressure signal is smaller than a threshold value, the observation signal as-is to the noise reduction processing unit.

According to an aspect of the present technology, the active state analysis unit may further include a first sensor analysis unit configured to determine non-attachment or noncontact, and the first sensor analysis unit may be configured to determine, in a case where it is determined in the first sensor analysis unit that the observation signal is smaller than a threshold value, non-attachment or noncontact.

According to an aspect of the present technology, a preprocessing unit configured to perform absolute value processing of a signal on a fluctuation component subjected to bandpass filter processing, as preprocessing of a signal to be input to the noise reduction processing unit may be further included.

According to an aspect of the present technology, the noise reduction processing unit may further include an adaptive filter processing unit, and

the noise reduction processing unit may be configured to calculate an error signal obtained by subtracting a reference signal of the adaptive filter processing unit as body motion noise from an observation signal.

According to an aspect of the present technology, the adaptive filter processing unit may be configured to add a transfer function of a band that is calculated from a pressing force signal difference between a pressing force change in skin, and a pressing force change between band materials, to a fluctuation component of a pressing force change that has been subjected to bandpass filter processing, and use as a reference signal.

Furthermore, the present technology provides a noise reduction processing method in biological information processing, including calculating an error signal obtained by subtracting body motion noise included in an observation signal from a first sensor configured to measure biological body affect as an observation signal,

on the basis of a body motion signal from a second sensor configured to measure a body motion change, and/or a pressure signal from a third sensor configured to measure a pressing force change in skin.

### Effects of the Invention

According to the present technology, it is possible to accurately reduce body motion noise included in an observation signal of biological information. Note that the effect described here is not necessarily limited, and may be any effect described in the present disclosure.

### BRIEF DESCRIPTION OF DRAWINGS

FIG. 1 is a diagram illustrating a strategic configuration of a system according to an embodiment of the present technology.

FIG. 2 is a diagram illustrating an example of attachment to a biological body of a biological information processing system according to the present embodiment.

FIG. 3 is a diagram illustrating an example of attachment to a biological body of a biological information processing system according to the present embodiment.

FIG. 4 is a conceptual diagram of a block diagram illustrating an internal configuration of the biological information processing system according to the present embodiment.

FIG. 5 is a diagram illustrating an example of an external appearance of the biological information processing system according to the present embodiment.

FIG. 6 is a schematic diagram illustrating an example of an external configuration of the biological information processing system according to the present embodiment.

FIG. 7 is a schematic diagram illustrating an example of an external configuration of the biological information processing system according to the present embodiment. FIG. 7 is a schematic diagram of a first sensor portion according to an embodiment of the present technology.

FIG. 8 is a schematic diagram illustrating an example of an external configuration of the biological information processing system according to the present embodiment. FIG. 8 is a schematic diagram of a first sensor portion according to an embodiment of the present technology.

FIG. 9 is a conceptual diagram of an overall block diagram of a biological information processing system according to a first embodiment of the present technology.

FIG. 10 is a diagram illustrating an example of a flowchart according to the first embodiment of the present technology.

FIG. 11 is a diagram illustrating an example of a flowchart according to the first embodiment of the present technology.

FIG. 12 is a diagram illustrating an example of a flowchart according to the first embodiment of the present technology.

FIG. 13 is a conceptual diagram of an overall block diagram of a biological information processing system according to a second embodiment of the present technology.

FIG. 14 is a conceptual diagram of a block diagram of a noise reduction processing unit of a biological information processing system according to a third embodiment of the present technology.

FIG. 15 is a block diagram illustrating a schematic configuration example of a biological processing apparatus according to an embodiment of the present technology.

FIG. 16 is a diagram illustrating a hardware configuration of an information processing apparatus according to an embodiment of the present technology.

### MODE FOR CARRYING OUT THE INVENTION

Hereinafter, a preferred mode for carrying out the present technology will be described with reference to the drawings.

The embodiment to be described below indicates an example of a representative embodiment of the present technology, and the scope of the present technology is not interpreted in a limited sense due to the embodiment. Note that the description will be given in the following order. Note that, in the drawings, the same or equivalent components or members are assigned the same reference numerals, and the redundant description will be appropriately omitted.

1. System Configuration

2. Internal Configuration of Biological Information Processing System

2-1. Sensor Unit 150

2-2. First Sensor Unit 151

2-3. Second Sensor Unit 152

2-4. Third Sensor Unit 153

2-5. Processing Unit 160

2-6. Noise Reduction Processing Unit 161

2-7. Active State Analysis Unit 162

3. External Configuration of Biological Information Processing System

4. Biological Information Processing Apparatus According to First Embodiment

4-1. First Active State Analysis Unit

4-2. Second Active State Analysis Unit

4-3. Third Active State Analysis Unit

5. Biological Information Processing Apparatus According to Second Embodiment

6. Biological Information Processing Apparatus According to Third Embodiment

7. Configuration Example of Analysis Apparatus

8. Hardware Configuration

1. System Configuration

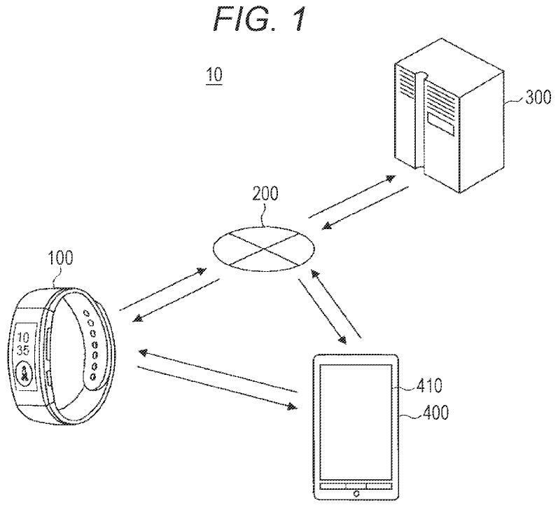

FIG. 1 is a diagram illustrating a strategic configuration of a system according to an embodiment of the present technology. Referring to FIG. 1, a system 10 includes a biological information processing apparatus 100. The system 10 may further include a server 300 connected to the biological information processing apparatus 100 via a network 200. Furthermore, the system 10 may include a terminal apparatus 400 different from the biological information processing apparatus 100.

The biological information processing system of the present embodiment is a system that detects information regarding the state of a biological body, and determines the affect of the biological body on the basis of the detected information. The biological information processing system of the present embodiment can be directly attached to a biological body for detecting information regarding the state of the biological body.

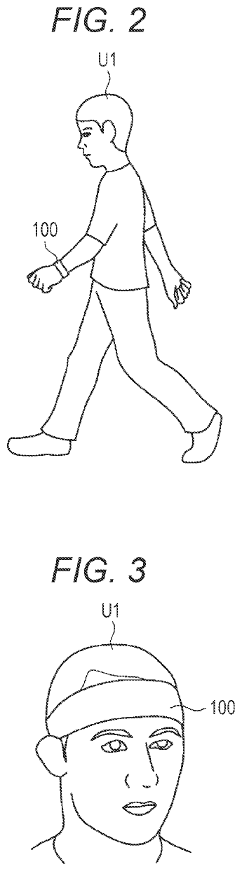

Specifically, the biological information processing system of the present embodiment is used as illustrated in FIGS. 2 and 3, for example, for determining the affect of a biological body. FIGS. 2 and 3 are diagrams each illustrating a state in which the biological information processing apparatus 100 of the present embodiment is attached to a biological body. In FIG. 2, a user U1 wears the biological information processing apparatus 100 having a wristband shape like a wristwatch type and the like, on his wrist. In FIG. 3, the user U1 wears the biological information processing apparatus 100 having a headband shape like a forehead contact type and the like, and being winded around his head. The biological information processing apparatus 100 recognizes biological information of the user U1 by detecting information for determining the affect of a biological body such as a sweat state, a pulse wave, a myoelectric potential, a blood pressure, or a body temperature of the user U1. On the basis of the biological information, a concentration state, an awake state, and the like of the user can be checked.

The description will be given of an example in which the biological information processing apparatus 100 is attached to an arm or a head, but the present technology is not limited to such an example.

For example, the biological information processing apparatus 100 may be implemented in a mode attachable to a part of a hand such as a wristband, a glove, a smart watch, or a ring. Furthermore, in a case where the biological information processing apparatus 100 contacts a part of a biological body such as a hand, the biological information processing apparatus 100 may have a configuration included in an object that can contact the user, for example. The biological information processing apparatus 100 may be provided on the surface or inside of a device that can contact the user, such as a mobile terminal, a smartphone, a tablet, a mouse, a keyboard, a handle, a lever, a camera, sporting equipment (golf club, tennis racket, archery, and the like), or a writing implement.

Furthermore, for example, the biological information processing apparatus 100 may be implemented in a mode attachable to a portion of a head of the user, such as a hat, an accessory, goggles, and glasses. Furthermore, the biological information processing system 100 may be provided on clothes such as sportswear, socks, underclothes, a protective gear, shoes, and the like.

A mode for implementing the biological information processing system is not specifically limited as long as the system is provided in such a manner that the system can contact the surface of a biological body. The biological information processing system needs not be in direct contact with the body surface of a biological body as long as information regarding the state of the biological body can be detected. For example, the biological information processing system may be in contact with the surface of the biological body via clothes, a detection sensor protective film, or the like.

Furthermore, the biological information processing system needs not be a wearable terminal, and may be a system that determines the affect of a biological body by performing information processing by another device on the basis of information detected by a sensor being in contact with the biological body. For example, in a case where a biological sensor is attached to an arm, a head, or the like of the user, the biological information processing system may output information acquired from the biological sensor, to another terminal such as a smartphone, and determine the affect of the biological body by performing information processing using the other terminal.

A biological sensor included in the biological information processing apparatus 100 detects biological information by contacting the surface of a biological body in the above-described various forms. Thus, influence attributed to a variation in contact pressure between a biological sensor and a biological body that is caused by a body motion of the biological body is easily exerted on a measurement result of the biological sensor. For example, biological data acquired from a biological sensor can include noise attributed to the body motion of a biological body. It is demanded to accurately determine the affect of a biological body from such biological information including noise.

The body motion of a biological body refers to all operation modes used when the biological body operates. For example, when the biological information processing apparatus 100 is attached to the wrist of the user U1, an operation of a biological body such as twisting of a wrist, flexing and stretching of fingers, and flexing and stretching of a part of fingers is included in the body motion. By such an operation of the user, contact pressure between the user U1 and a biological sensor included in the biological information processing apparatus 100 can vary.

The biological information processing apparatus 100 according to the present embodiment preferably includes a second sensor and/or a third sensor for improving the accuracy of information obtained by a biological sensor. The second sensor is configured to detect a body motion change of a biological body. The third sensor is configured to detect a pressure change of a biological body in a region corresponding to a detection region of the biological sensor. The biological information processing system according to the present embodiment can accurately reduce body motion noise from an observation signal (false signal) detected by the biological sensor, using a detected body motion signal and/or pressure signal. By correcting the observation signal in this manner, an error signal (biological information data) with improved accuracy can be obtained.

2. Internal Configuration of Biological Information Processing System

<2-1. Sensor Unit 150>

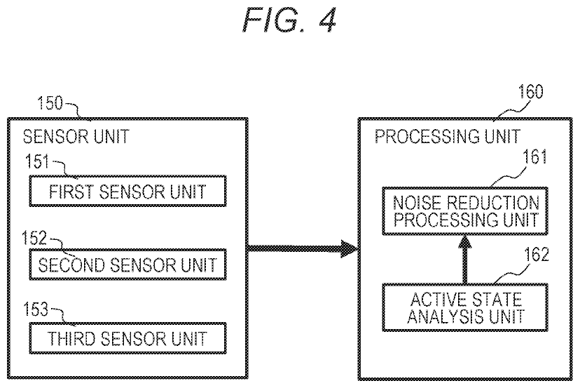

FIG. 4 illustrates a conceptual diagram of a block diagram illustrating an internal configuration of the biological information processing system according to the present embodiment, but the present embodiment is not limited to this.

As illustrated in FIG. 4, the biological information processing system of the present embodiment includes a sensor unit 150 and a processing unit 160.

The sensor unit 150 includes at least a first sensor unit 151 for measuring biological information, and a sensor unit that can measure at least a body motion change or a pressing force change in the skin. Each sensor can output each piece of sensor information measured by each sensor, to each component such as a processing unit, as each signal. The sensor unit that can measure the change is at least either a second sensor unit 152 that measures a body motion change, or a third sensor unit 153 that measures a pressing force change in the skin. The sensor unit 150 desirably includes the second sensor unit 152 and the third sensor unit 153 in such a manner that body motion noise can be accurately reduced (refer to FIG. 4).

The processing unit 160 includes at least a noise reduction processing unit 161 that calculates an error signal obtained by subtracting body motion noise included in an observation signal. Moreover, the sensor unit 150 desirably includes an active state analysis unit 162 that determines a reference signal for accurately subtracting body motion noise on the basis of a signal from the second sensor and/or third sensor (refer to FIG. 4).

<2-2. First Sensor Unit 151>

The first sensor unit 151 is configured to have a function of detecting information for determining the affect of a biological body.

For example, the first sensor unit 151 may be a sweat sensor. The sweat sensor is a sensor that detects sweat secreted from a sweat gland of the skin (for example, Eccrine sweat gland). By the sweating, the skin enters an electricity conducting state. Thus, the sweat sensor can detect sweating by acquiring an Electro Dermal Activity (EDA) state of the skin.

The sweat sensor includes a single electrode pair or a plurality of electrode pairs. The electrode pair preferable has a configuration of contacting the contact of the user and contacting a wrist portion. A current flowing between an electrode pair may be either a direct current or an alternating current. The sweat sensor may include a voltage/power supply unit for flowing a current in the skin from the electrode pair, a current voltage conversion unit, an amplification unit for amplifying a skin conductance, a filter unit that performs filter processing of an amplification signal, and an analog/digital (A/D) conversion unit. The sweat sensor can output an observation signal of a skin conductance (SC signal) to each component.

The sweat sensor has been exemplified above as the first sensor unit 151, but the type of the sensor of the first sensor unit 151 is not specifically limited as long as information for determining the affect of a biological body can be detected. Aside from the sweat sensor, for example, the biological sensor may be a pulse wave sensor, a heartbeat sensor, a blood pressure sensor, a body temperature sensor, or the like.

By such a biological sensor, biological information of the user can be acquired. The one or more biological sensors can be provided in the biological information processing system 100. Biological information acquired by the biological sensor is output to the processing unit 160 as an observation signal.

<2-3. Second Sensor Unit 152>

The second sensor unit 152 is configured to have a function of detecting information for determining a body motion change of a biological body. The type of the sensor of the second sensor unit 152 is not specifically limited as long as information for determining a body motion change of a biological body can be detected.

For example, the second sensor unit 152 may be an acceleration sensor or an angular velocity sensor. The acceleration sensor may use, for example, a mechanical displacement measurement method, a vibration-based method, an optical method, a semiconductor method, or the like. Furthermore, the acceleration sensor includes a single-axis sensor, a biaxial sensor, and a triaxial sensor depending on the number of axes to be detected, but the acceleration sensor is not specifically limited. For example, a triaxial acceleration sensor is one type of a Micro Electro Mechanical Systems (MEMS) sensor that can measure accelerations in three direction along XYZ axes, by one device.

By such a body motion change sensor, body motion change information regarding biological information of the user can be acquired. The one or more body motion change sensors can be provided in the biological information processing system 100. Body motion change information acquired by the body motion change sensor is output to the processing unit 160 as a body motion signal.

<2-4. Third Sensor Unit 153>

The third sensor unit 153 has a function of detecting a pressure change in a region corresponding to a detection region of the first sensor unit 151. The type of the sensor of the third sensor unit 153 is not specifically limited as long as the sensor can generally detect a pressure. The third sensor unit 153 is only required to be an element or the like (piezoelectric element or the like) that varies in voltage, current, resistance depending on the pressure, for example, and may be a pressure-sensitive conductive elastomer obtained by mixing conductive material with high-polymer material, for example.

The pressure-sensitive conductive elastomer deforms in accordance with a pressure change, and conductive material elements included in the pressure-sensitive conductive elastomer start to contact each other. The conductivity in the pressure-sensitive conductive elastomer is therefore enhanced, and electric resistibility declines. Based on the difference in electric resistance value, the pressure-sensitive conductive elastomer can detect a pressure.

The third sensor unit 153 performs detection of a region corresponding to the region detected by the first sensor unit 151. The region corresponding to the region detected by the first sensor unit 151 may be a region at least partially overlapping a region in which the first sensor unit 151 is disposed. By the third sensor unit 153 detecting the region at least partially overlapping the region in which the first sensor unit 151 is disposed, it is possible to correct first sensor information more accurately.

Furthermore, the region corresponding to the region detected by the first sensor unit 151 may be a region including the entire region in which the first sensor unit 151 is disposed. The third sensor unit 153 can thereby detect a body motion pressure change while encompassing the detection region of the first sensor unit 151. It is therefore possible to detect a body motion pressure change related to the first sensor unit 151.

The detection region is not limited to the above-described region, and the detection region of the third sensor unit 153 may be appropriately set in accordance with the detection region of the first sensor unit 151. For example, it becomes easier to detect a region in which the detection region of the third sensor unit 153 deviates from the detection region of the first sensor unit 151. Therefore, in a case where the detection region of the third sensor unit 153 is excessively larger than the detection region of the first sensor, there is a possibility that the detection accuracy of a body motion pressure change related to the first sensor unit 151 declines. Thus, the detection region of the third sensor unit 153 may be appropriately set in accordance with a positional relationship between the first sensor unit 151 and the third sensor unit 153, a region area, or the like.

Moreover, the region corresponding to the detection region of the first sensor unit 151 may be a region near the region in which the first sensor unit 151 is disposed, and needs not necessarily include a portion overlapping the region in which the first sensor unit 151. By detecting a body motion pressure change near the region in which the first sensor unit 151, it is possible to approximately acquire a body motion pressure change related to the region detected by the first sensor unit 151, and correct first sensor information.

Furthermore, the second sensor unit 152 and/or the third sensor unit 153 may be calibrated at a predetermined timing. By the second sensor unit 152 being calibrated, it is possible to detect a body motion change of a biological body more accurately. Furthermore, by the third sensor unit 153 being calibrated, it is possible to detect a body motion pressure of a biological body more accurately. Furthermore, by accumulating data of information from these sensors, a correction value for correcting first sensor information from the data analysis result may be calculated and the correction value may be updated in real time. By using the correction value, it is possible to calculate an error signal obtained by subtracting body motion noise included in first sensor information, more accurately.

For example, when the user wears the biological information processing apparatus 100, the second sensor and/or the third sensor may be calibrated. From when the user wears the biological information processing apparatus 100, a contact pressure change between the biological body and the biological information processing apparatus 100, and a body motion change of the biological body start to occur. For detecting a body motion change and a body motion pressure change of the biological body, a mere contact pressure change between the biological body at rest and the biological information processing apparatus 100, and a body motion change of the biological body can be body motion noise. Thus, by performing calibration when the user wears the biological information processing apparatus 100, it is possible to calculate an error signal obtained by subtracting body motion noise included in first sensor information, more accurately.

Meanwhile, stimulus to a human includes a high-order route passing through an amygdala via sensory thalamus/sensory cortex, and a low-order route passing through an amygdala via from sensory thalamus. In the high-order route, stimulus is analyzed and delivered to an amygdala, which takes time. On the other hand, in the low-order route, processing of high-order brain cortex is omitted and prompt evaluation of stimulus can be performed. It is known that the amygdala causes body response such as an affective response, an autonomic response, and hormonal secretion, through hypothalamus/autonomic nerve. The sweat gland existing under the skin is linked with an autonomic nerve, and develops sweating in accordance with stimulus.

Sweating is broadly divided into thermal sweating for controlling body temperature under the hot environment or at the time of exercise, for example, mental sweating caused when mental stimulus such as mental tension or emotional fluctuations is received, gustatory sweating caused, for example, when hot food or spicy food is eaten, and the like.

As a method for measuring a skin state change caused by sweating on the body surface, there is a method of arranging at least two or more electrodes on the body surface, and measuring an impedance change or conductance change between the electrodes that is cause by applying a voltage or a current between the electrodes.

Meanwhile, it is said that sweat glands that often cause mental sweating being an affective response exist at limited positions, and such sweat glands often exist at fingertips, palms, and feet bottoms and rarely exist at wrist positions. Mental sweating can be appropriately measured by measuring fingertips, palms, and feet bottoms, but behaviors in daily life are restricted, which places a heavy burden on a subject. On the other hand, measurement of a wrist position hardly influences behaviors in daily line and is preferable for sweating measurement. As a shape of a device that measures sweating at a wrist position, a wristband type device and a watch type device are conceivable. Electrodes of a wristband type sweat sensor are disposed on the inside of a wristband.

However, mental sweating measurement at a wrist position in daily life has the following problems. For example, a skin conductance change caused by thermal sweating under the hot environment or at the time of exercise becomes noise. As a measure against noise caused by thermal sweating at the time of exercise, Reference Literature 1 (Predicting students' happiness from physiology, phone, mobility, and behavioral data) proposes a method of mounting an acceleration sensor on a wristband and normalizing a skin conductance measurement value SC using a calculation formula of an acceleration signal intensity.

However, while normal operations in daily life do not involve a strenuous movement such as exercise, the operations often involve the movement of a part of a body like a movement of a finger or a wrist, such as routine operations including washing of a face and brushing of teeth, eating, a PC operation, and a smartphone operation, for example. Because a part of a body (for example, a shape of an arm) is moved, even when being attached to a biological information processing system, an acceleration sensor sometimes fails to perform accurate detection. Furthermore, in normal operations in daily life, a part of a body (for example, a shape of an arm) to which the biological information processing system is attached changes, and this change influences a contact portion between the sensor and a biological body and causes body motion noise. For example, in the case of a wristband type biological information processing system, a skin conductance change caused by a change in the shape of an arm and a pressing force change between electrodes and the skin becomes body motion noise. In Reference Literature 1 described above, because a skin conductance change caused by a pressing force change is not considered, there are problems such as a problem of noise erroneously detected as a skin conductance value caused by mental sweating.

The present disclosure can also provide a signal processing method and a processing apparatus that prevent erroneous detection of skin conductance measurement accompanying mental sweating, even in a case where noise is generated by a skin conductance change attributed to a pressing force change between electrodes and the skin that is caused by an operation in daily life, in skin conductance measurement accompanying mental sweating in daily life.

<2-5. Processing Unit 160>

The processing unit 160 includes at least the noise reduction processing unit 161 (refer to FIG. 4). The processing unit 160 may further include the active state analysis unit 162 together with the noise reduction processing unit 161. The processing unit 160 is configured to acquire sensor information by the sensor unit 150. The processing unit 160 is configured to have a function of correcting first sensor information using second sensor information and/or third sensor information. The noise reduction processing unit 161 is configured to calculate an error signal obtained by subtracting body motion noise included in an observation signal from the first sensor unit 151, on the basis of a body motion signal from the second sensor unit 152, and/or a pressure signal from the third sensor unit 153.

<2-6. Noise Reduction Processing Unit 161>

The noise reduction processing unit 161 is configured to acquire first sensor information by the first sensor unit 151. The first sensor information is information for determining the affect of a biological body. For example, in the case of a sweat sensor, the first sensor information includes information regarding a timing at which the generation of sweating starts, information regarding an amount of sweating and the like, and the like.

The noise reduction processing unit 161 can acquire second sensor information by the second sensor unit 152, and/or acquire third sensor information by the third sensor unit 153. The second sensor information is information regarding a body motion change of a biological body. The second sensor information includes, for example, a direction in which a body is moved, a size (body motion value) of the movement, a time from the start to the end of the movement, and body motion change information regarding a body motion change and the like. Furthermore, the third sensor information includes information regarding body motion pressure of a biological body that is based on a pressing force change between the sensor and the human skin that is caused by a body motion. The third sensor information includes, for example, a body motion pressure value of a body motion pressure change detected by the third sensor unit 153 when a biological body moves, timings at which the change starts and ends, an elapsed time of the change, and pressure change information regarding a pressure change and the like. Furthermore, the biological information processing apparatus 100 may be further provided with a center information acquisition unit that acquires information from the sensor unit 150, and various types of information may be transmitted from the center information acquisition unit to the noise reduction processing unit 161.

The noise reduction processing unit 161 is configured to have a function of subtracting body motion noise from the first sensor information, using either or both of the second sensor information or the third sensor information. For example, in a case where the first sensor unit 151 is a sweat sensor, the first sensor unit 151 may be configured to have a function of correcting the first sensor information by removing body motion noise and the like that are included in information obtained by the sweat sensor.

The noise reduction processing unit 161 can identify body motion noise included in the first sensor information, on the basis of a determination result of an active state obtained by the active state analysis unit 162, and perform correction processing of removing the noise from first sensor information. Furthermore, the noise reduction processing unit 161 can also determine that body motion noise is not included, on the basis of a determination result of an active state obtained by the active state analysis unit 162, and directly perform transmission without removing body motion noise from the first sensor information. In a case where the noise is not included, biological sensor information may be transmitted from another processing unit other than the noise reduction processing unit 161 to the next step.

Furthermore, the noise reduction processing unit 161 can also notify the user that the biological information processing system 100 is not attached or not bonded, on the basis of a determination result of an active state obtained by the active state analysis unit 162. The processing unit 160 may perform such a user notification.

<2-7. Active State Analysis Unit 162>

The active state analysis unit 162 is configured to have a function of analyzing an active state of a biological body on the basis of each sensor information from each sensor unit (specifically, each signal of observation signal, body motion signal, or pressure signal).

The active state analysis unit 162 is configured to have a function of determining an attachment status of the biological information processing system and/or an active state of a biological body on the basis of the sensor information. Specifically, the active state analysis unit 162 can determine whether or not the system is not attached or the first sensor is not in contact, on the basis of the sensor information, as for an attachment status of the biological information processing system. Furthermore, the active state analysis unit 162 can determine a status of an active state of a biological body to be an active state, a quasi-rest state, or a rest state on the basis of the sensor information.

The active state includes, for example, a state in which a body moves drastically like an exercise, stretching, or the like. More specifically, the active state includes a state in which an arm moves drastically, and the like. The quasi-rest state includes, for example, a state in which a part of a body moves small like a smartphone work, a PC work, or the like. More specifically, the quasi-rest state includes a state in which a finger or a wrist is moving at the time of an operation of a smartphone or a PC, and the like. The rest state includes, for example, a state in which a biological body hardly moves like rest, short sleep, or the like.

The active state analysis unit 162 is configured to have a function of determining body motion noise (specifically, reference signal) from second sensor information (specifically, body motion signal) or third sensor information (specifically, pressure signal) on the basis of the above-described analysis result. Specifically, in a case where the active state analysis unit 162 determines that an analysis result indicates the active state, the active state analysis unit 162 determines the second sensor information (specifically, body motion signal) as body motion noise. In a case where the active state analysis unit 162 determines that an analysis result indicates the quasi-rest state, the active state analysis unit 162 determines the third sensor information (specifically, pressure signal) as body motion noise. In a case where the active state analysis unit 162 determines that an analysis result indicates the rest state, the active state analysis unit 162 determines that body motion noise is not included. Furthermore, the active state analysis unit 162 can also determine that the biological information processing system is not attached or the first sensor is not in contact, from the first sensor information.

Furthermore, each sensor information is desirably processed into fluctuation components using a bandpass filter or the like.

When each state is determined, as necessary, each threshold value (for example, contact analysis threshold value, body motion analysis threshold value, pressing force analysis threshold value, and the like) may be set in the active state analysis unit 162. The active state analysis unit 162 may be configured to analyze each sensor information and set a threshold value from the analysis result, or may be configured to set a threshold value on the basis of an input from the user or the like. Furthermore, the user may determine right and wrong and input a determination result of an active state analysis, and the active state analysis unit 162 may be configured to correct a threshold value on the basis of the user determination result.

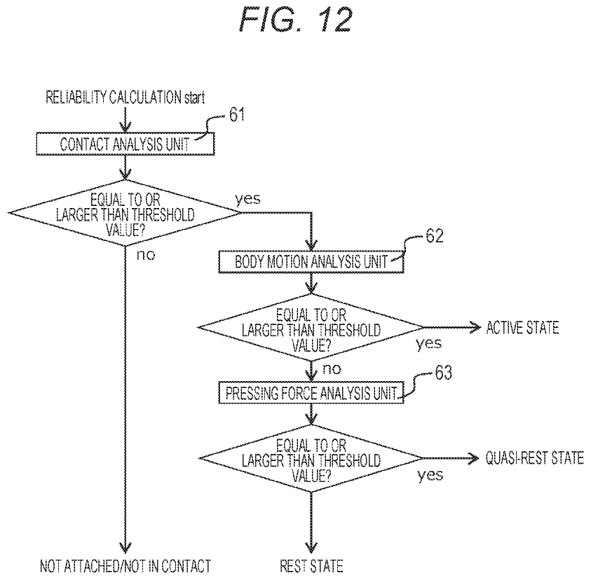

The active state analysis unit 162 is preferably configured to perform active state analysis in the order of first sensor analysis (contact analysis), second sensor analysis (body motion analysis), and third sensor analysis (pressing force analysis) (for example, refer to FIG. 12 and the like as described later). In a case where it is determined in the second sensor analysis (body motion analysis) that a body motion signal of second sensor information is equal to or larger than a threshold value, the active state analysis unit 162 outputs the body motion signal to the noise reduction processing unit 161 as a reference signal. Furthermore, in a case where it is determined in the second sensor analysis (body motion analysis) that the body motion signal of the second sensor information is smaller than the threshold value, and subsequently, it is determined in third sensor analysis (pressing force analysis) that a pressing force signal of third sensor information is equal to or larger than a threshold value, the noise reduction processing unit 161 outputs the pressing force signal as a reference signal. Furthermore, in a case where it is determined in the third sensor analysis (pressing force analysis) that the pressing force signal of the third sensor information is smaller than the threshold value, the noise reduction processing unit 161 does not output a reference signal or outputs nonexistence of a reference signal. Note that, by the user setting that “a body is in an inactive state”, “a body is not in a quasi-rest state”, or the like, the second sensor analysis or the third sensor analysis (body motion analysis or pressing force analysis) can be omitted or skipped (for example, refer to FIGS. 10 and 11 as described later).

Furthermore, the active state analysis unit 162 may include the above-described first sensor analysis unit that determines noncontact and the like, the above-described second sensor analysis unit that determines an active state, or the above-described third sensor analysis unit that determines a quasi-active state. Furthermore, the active state analysis unit 162 may further include a threshold value processing unit including each threshold value for analyzing an active state. Furthermore, the threshold value processing unit may be included in the first sensor analysis unit (contact analysis unit), the second sensor analysis unit (body motion analysis unit), the third sensor analysis unit (pressing force analysis unit), or another unit.

<Noise Reduction Processing Method in Biological Information Processing System>

An example of an operation of the biological information processing system 100 in the present technology will be described below, but the operation is not limited to this. Noise reduction processing of biological information can be thereby performed.

A noise reduction processing method in biological information processing of the present technology can calculate an error signal obtained by subtracting body motion noise included in an observation signal from a first sensor that measures biological body affect as an observation signal, on the basis of a body motion signal from a second sensor that measures a body motion change, and/or a pressure signal from a third sensor that measures a pressing force change between the skin.

Moreover, the noise reduction processing method preferably includes performing active state analysis in the order of the observation signal, the body motion signal, and/or the pressure signal, and determining body motion noise on the basis of the analysis result. The noise reduction processing method preferably uses a wristband type sweat sensor, and can thereby reduce body motion noise of the sweat sensor.

Moreover, a body motion noise reduction processing method of a sweat sensor in the present technology can analyze an active state in the sweat sensor using a pressure sensor that measures a pressing force change between electrodes for skin conductance measurement and the skin, and an acceleration sensor that measures a body motion change. After analyzing the active state, the method can reduce body motion noise superimposed on skin conductance, using an acceleration signal and a pressure signal.

Moreover, the noise reduction processing method in the present technology can determine an active state from a skin conductance signal, an acceleration signal, and a pressure signal, and thereby reduce body motion noise of the sweat sensor.

Moreover, the noise reduction processing method of the present technology can reduce body motion noise superimposed on skin conductance, using an adaptive filter that uses fluctuation components in a pressure signal subjected to bandpass filter processing, as a reference signal.

The noise reduction processing method of the present technology can use a signal obtained by performing absolute value processing on fluctuation components in the pressure signal subjected to bandpass filter processing, and thereby reduce body motion noise in the sweat sensor.

The noise reduction processing method of the present technology can preliminarily obtain and store a transfer function of a band from signals of a pressing force change on an electrode surface and a pressure change in the band. Moreover, the method can use, as a reference signal of an adaptive filter, a signal obtained by convoluting the transfer function into fluctuation components obtainable after bandpass filter processing. Body motion noise of the sweat sensor can be thereby reduced.

3. External Configuration of Biological Information Processing System

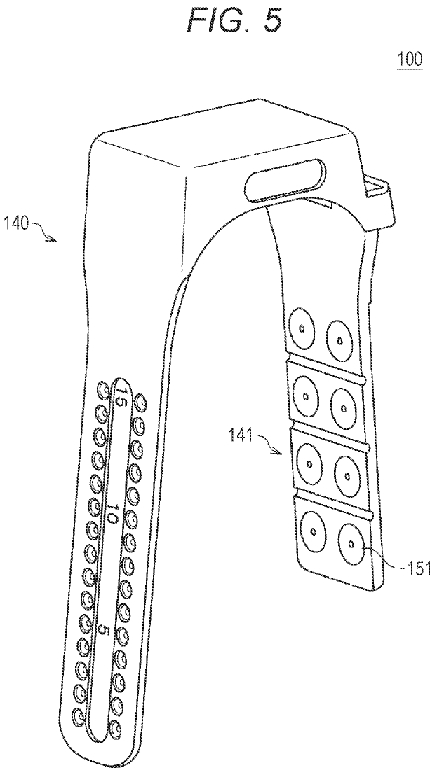

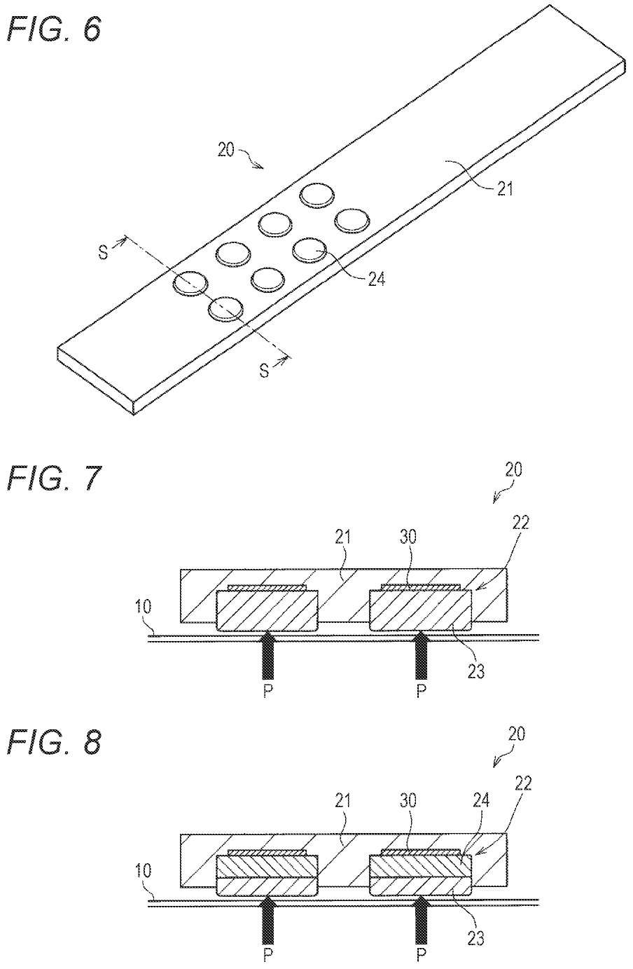

An overview of an external configuration of the biological information processing system will be described with reference to FIGS. 5 to 7, but the present disclosure is not limited to this. FIG. 5 is a diagram illustrating an example (wristband type) of an external appearance of the biological information processing apparatus 100. FIGS. 6 and 7 are schematic diagrams each illustrating an example of a configuration of a sensor unit in the biological information processing apparatus 100 and a neighborhood portion thereof.

As illustrated in FIG. 5, the biological information processing apparatus 100 includes a wristwatch-type biological sensor module 140, and the module 140 may include the second sensor unit 152 (for example, acceleration sensor), the processing unit 160, and the like. When the biological information processing apparatus 100 is attached to the wrist of the user, a body motion change caused by an operation of the wrist can be detected by the acceleration sensor.

A biological sensor 151 is built into a wristband 141 with being exposed to the surface of the wristband 141. The wristband 141 has a function of supporting the biological sensor 151. The wristband 141 has a shape extended in one direction. Then, by winding the wristband 141 around a biological body like a wristwatch, the biological information processing system 100 can be attached. The material of the wristband 141 may be rubber, leather, organic resin, or the like, and elastic material is preferable for easy attachment. A plurality of pairs of biological sensors 151 is disposed at equal intervals in the wristband extending direction on the biological body side of the wristband 141. The shape of exposed portion of the biological sensor 151 may have a circular shape. In this example, the description has been given of an example in which the shape of the biological sensor 151 is circular shape, but the shape is not specifically limited, and may have a shape such as an ellipse, a rectangle, or a polygon.

Furthermore, the number of biological sensors 151 provided on the wristband 141 is not specifically limited, and one or more biological sensors 151 can be provided. A sensor that is different from the biological sensor 151, and for detecting the deformation of the wristband 141, force exerted on the wristband, and a shape change of the wristband 141 is provided between the biological sensor 151 and the wristband 141. For example, the third sensor unit 153 (for example, pressure sensor) is provided between the exposed surface of the biological sensor 151 and the wristband 141. When the biological information processing system 100 is attached to the wrist of the user, a body motion pressure change caused by an operation of the wrist can be detected by the pressure sensor.

Using schematic diagrams schematically illustrating the biological sensor 151 provided on the wristband 141, a state in which the biological sensor 151 and a pressure sensor 153 in the biological information processing apparatus 100 function will be described with reference to FIGS. 7 and 8.

On a wristband 21 included in a biological information processing apparatus 20, a pair of sensor units 22 is provided at an equal interval in the extending direction of the wristband 21. FIG. 7 is a cross-sectional diagram taken along an S-S line in FIG. 6, and illustrates a state in which the wristband 21 is winded around a surface of a biological body 10 (for example, skin). The sensor units 22 are built in the wristband 21 attached onto the surface of the biological body 10. The sensor units 22 each include a biological sensor 23 and a pressure sensor 30, and the sensor unit 22 and the wristband 21 have a three-layer structure. The three-layer structure is disposed in such a manner that the biological sensor 23, the pressure sensor 30, and the wristband 21 are stacked in this order from the biological body 10 side. A region in which the pressure sensor 30 is disposed overlaps the inside of a region in which the biological sensor 23 is disposed, and the pressure sensor 30 is disposed immediately above the biological sensor 23 in an opposite direction to the biological body side.

Furthermore, the biological information processing apparatus 20 illustrated in FIG. 8 is a modified example of the biological information processing apparatus illustrated in FIG. 7. FIG. 8 is a cross-sectional diagram taken along an S-S line in FIG. 6, and the description of the same configurations as those in the example illustrated in FIG. 7 will be appropriately omitted. The sensor units 22 and the wristband 21 of the wristband 21 in FIG. 8 have a four-layer structure disposed in such a manner that the biological sensor 23, a deformable member 24, and the wristband 21 are stacked in this order from the biological body 10 side. The deformable member 24 is disposed between the biological sensor 23 and the pressure sensor 30. It is preferable that the deformable member 24 formed by high-polymer material, and is formed by material deformable by pressure and restorable to an original shape by the release of pressure. Examples of material of the deformable member 24 include rubber, silicone rubber, organic resin, and the like. The deformable member 24 may include material having a larger deformation amount than the wristband 21 in the case of being pressed by the same pressure. In the present technology, importance is basically placed on use of acceleration information of a body motion and pressing force information between a sensor and a human skin that is based on a body motion. Thus, a measurement method of each sensor and a sensor apparatus are not specifically limited, which is advantageous.

In the biological information processing system having the above-described configuration, a sensor electrode of the biological sensor 23 is displaced in an arrow direction by pressing force P from the side of an attachment surface represented by a skin of a biological body or the like. The pressure is transmitted to the pressure sensor 30 while the displacement is generated in the entire wristband 21. The pressure applied to the sensor electrode of the biological sensor 23 can be thereby detected.

Furthermore, a state in which the surface of the biological body and the pressing force surface of the biological sensor 23 are parallel can be obtained. With this configuration, by the pressing force surface and the surface of the biological body becoming parallel, pressing force on the surface of the biological body can be correctly transmitted. Thus, it is possible to improve the detection accuracy of the pressure sensor 30.

Furthermore, the biological sensor may be formed into a protruding shape (not illustrated) protruding upward from the contact surface of the wristband 141 with the biological body. A projection portion of the protruding shape is formed to project straight upward at the center of the biological sensor toward the surface of the wristband 141 that is opposite to the biological body. In the biological sensor, various circular configurations are disposed with the same central axis as the projection shape, from the configuration on the contact surface toward an end portion of the projection portion. Therefore, when a pressure sensor detects pressing force on the wristband attached surface side, it is possible to effectively detect pressure while limiting a pressure direction of a desired location.

Furthermore, in a case where the deformable member 24 is included, due to a difference in hardness between the deformable member 24 and a main body of the wristband 21 that uses material with higher hardness than the deformable member 24, the deformable member 24 having lower hardness is displaced more. By the force generated as reactive force of compression deformation in the deformable member 24 being transmitted to the pressure sensor 30, the pressure applied to the sensor electrode of the biological sensor 23 can be detected.

4. Biological Information Processing Apparatus According to First Embodiment

Hereinafter, the biological information processing apparatus 100 according to the first embodiment of the present technology will be described, but the present technology is not limited to this.

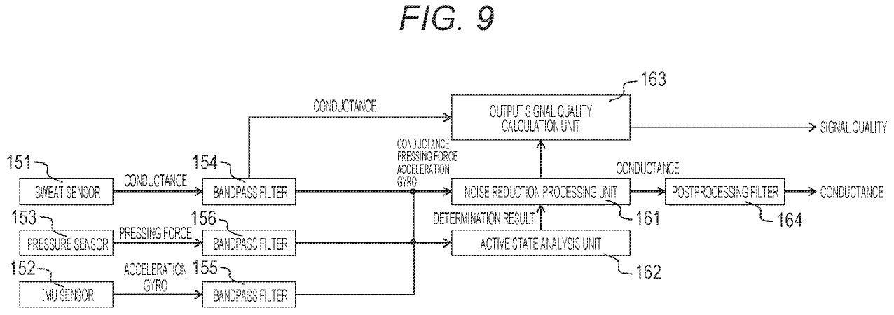

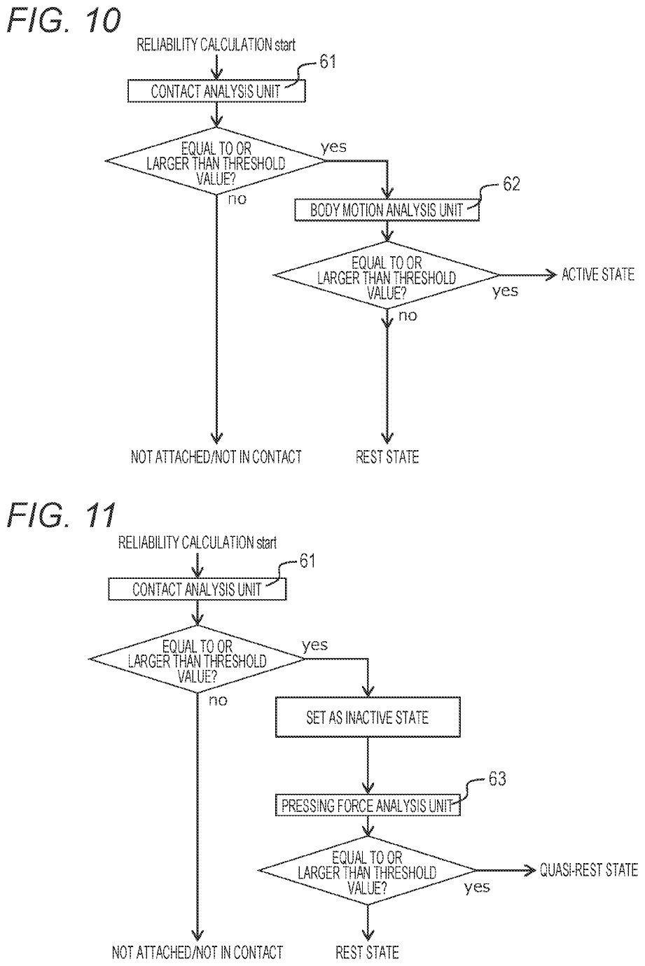

As the first embodiment of the present technology, FIG. 9 illustrates an overall block diagram for reducing body motion noise superimposed on skin conductance, on the basis of a sweat sensor being a first sensor, an acceleration sensor being a second sensor, and/or a pressure sensor being a third sensor. The processing unit 160 includes the active state analysis unit 162, the active state analysis unit 162 includes a first sensor analysis unit 61, and includes either or both of the second sensor analysis unit 62 or the third sensor analysis unit 63.

In a case where the first sensor analysis unit 61 is a sweat sensor, the first sensor analysis unit 61 is preferably a contact analysis unit 61. Furthermore, in a case where the second sensor analysis unit 62 is an acceleration sensor, the second sensor analysis unit 62 is preferably a body motion analysis unit 62. Furthermore, in a case where the third sensor analysis unit 63 is a pressure sensor, the third sensor analysis unit 63 is preferably a pressing force analysis unit 63.

The description will be given of an example in which the first sensor unit 151 is a sweat sensor, but the first sensor unit 151 is not limited to this. The sweat sensor 151 is an example of a sensor attached to or brought into contact with an individual, for example, and has a function of detecting information (biological information) for determining the affect of a biological body of the user. The sweat sensor 151 being a first sensor measures the affect of the biological body as an observation signal. The skin conductance measured by the sweat sensor 151 is transmitted to the processing unit 160 as an observation signal.

The first sensor analysis unit 61 is configured to receive an observation signal input from the first sensor unit 151 that measures the affect of the biological body. In a case where the first sensor unit 151 is a sweat sensor, the measured skin conductance is input to the first sensor analysis unit 61 as an observation signal. The contact analysis unit 61 is configured to determine whether or not an observation signal is equal to or larger than a threshold value, and in a case where the observation signal is equal to or larger than a threshold value, determine that the biological body and the first sensor are in contact. The contact analysis unit 61 is configured to determine that the biological information processing apparatus is not attached or the first sensor is not in contact, in a case where the observation signal is smaller than the threshold value.

A body motion signal from the second sensor unit 152 that measures a body motion change is input to the second sensor analysis unit 62. The description will be given of an example in which the second sensor is an acceleration sensor, but the second sensor is not limited to this, and may be a gyro sensor or the like. The second sensor analysis unit 62 is configured to determine whether or not a body motion signal is equal to or larger than a threshold value, and in a case where the body motion signal is equal to or larger than a threshold value, determine that a biological body is in an active state. Moreover, in a case where the second sensor analysis unit 62 determines that a biological body is in an active state, the second sensor analysis unit 62 may transmit a body motion signal to the noise reduction processing unit 161 as a reference signal of body motion noise. Furthermore, the second sensor analysis unit 62 is configured to determine that a biological body is not in an active state, in a case where a body motion signal is smaller than a threshold value.

The second sensor analysis unit 62 may include a norm value processing unit and a maximum value filter unit. The norm value processing unit is a configured to perform norm value processing on fluctuation components extracted by a bandpass filter and input as a body motion signal. The maximum value filter unit is configured to perform maxim filter processing on a signal subjected to norm value processing. With this configuration, the second sensor analysis unit calculates a result value of second sensor analysis. It is preferable that the second sensor analysis unit 62 further includes a buffer for acquiring only a signal value at a time interval requiring the maximum value filter unit. Furthermore, the second sensor analysis unit 62 may further include a bandpass filter unit (hereinafter, will also be referred to as a BPF unit), or may use fluctuation components subjected to BPF processing in another unit, as a body motion signal.

It is more preferable that the second sensor analysis unit 62 includes a BPF unit, a norm value processing unit, a buffer, and a maximum value filter unit. With this configuration, a body motion signal from an acceleration sensor sequentially passes through the BPF unit, the norm value processing unit, the buffer, and the maximum value filter unit, and a more accurate value of a body motion analysis result can be obtained.

The second sensor analysis unit 62 can determine an active state from a body motion signal from a second sensor. In a case where an acceleration sensor is a triaxial acceleration sensor, values from a norm value to a norm value of a body motion signal are input to a maxim filter unit as a body motion signal. At this time, the maximum value filter unit may acquire, via the buffer, only a signal value at a required time interval. The body motion signal subjected to maximum value filter processing performed by the maximum value filter unit is used in the second sensor analysis unit 62 for determination as to whether or not a biological body is in an active state.

A pressing force signal from the third sensor unit 153 that measures a pressing force change is input to the third sensor analysis unit 63. The description will be given of an example in which the third sensor is a pressure sensor, but the third sensor is not limited to this example. The third sensor analysis unit 63 is configured to determine whether or not a pressing force signal is equal to or larger than a threshold value, and in a case where the pressing force signal is equal to or larger than a threshold value, determine that a biological body is in a quasi-rest state. In a case where the third sensor analysis unit 63 determines that a pressing force signal indicates the quasi-rest state, the third sensor analysis unit 63 may transmit the pressing force signal to the noise reduction processing unit 161 as a reference signal of body motion noise. Furthermore, the third sensor analysis unit 63 is configured to determine that a biological body is in a rest state, in a case where a pressing force signal is smaller than a threshold value. In a case where the third sensor analysis unit 63 determines that a pressing force signal indicates the rest state, the third sensor analysis unit 63 may transmit nonexistence of a reference signal of body motion noise to the noise reduction processing unit 161.

The third sensor analysis unit 63 may include a maxim filter unit. It is preferable that the third sensor analysis unit 63 includes a BPF unit, a differential absolute filter unit, a buffer, and a maximum value filter unit. With this configuration, a pressing force signal from a pressing force sensor sequentially passes through the BPF unit, the differential absolute filter unit, the buffer, and the maximum value filter unit, and a more accurate value of a pressing force analysis result can be obtained.

The third sensor analysis unit 63 can determine a quasi-rest state from a pressing force signal from a third sensor. In a case where the third sensor is a pressure sensor, a pressing force signal is input to the maxim filter unit. At this time, the maximum value filter unit may acquire, via the buffer, only a signal value at a required time interval. The pressing force signal subjected to maximum value filter processing performed by the maximum value filter unit is used in the third sensor analysis unit 63 for determination as to whether or not a biological body is in a quasi-rest state.

The first embodiment of the present technology will be described in more detail with reference to FIGS. 9 to 12.

The biological information processing apparatus of the first embodiment includes a noise reduction processing unit that calculates an error signal obtained by subtracting body motion noise included in an observation signal from the sweat sensor 151 that measures biological body affect as an observation signal. The processing unit 161 is configured to calculate an error signal obtained by subtracting body motion noise included in an observation signal, on the basis of a body motion signal from the acceleration sensor 152 that measures a body motion change, and/or a pressure signal from the pressure sensor 153 that measures a pressing force change between the skin. The noise reduction processing unit 161 is configured to regard either the body motion signal or the pressure signal as a reference signal, and calculate an error signal by subtracting body motion noise from the observation signal, using the reference signal.

It is desirable that, in the first embodiment, a bandpass filter unit 154, a bandpass filter unit 155, or a bandpass filter unit 156 that extracts fluctuation components from the signal using a bandpass filter is further included. The BPF unit 154 is configured to extract fluctuation components from skin conductance. The BPF unit 155 is configured to extract fluctuation components from a body motion signal. The BPF unit 156 is configured to extract fluctuation components from a pressing force signal. It is desirable that fluctuation components are extracted from each signal by a corresponding BPF unit. Therefore, highly-accurate biological information can be obtained

It is desirable that, in the first embodiment, an output signal quality calculation unit 163 that determines a reduction state of body motion noise on the basis of a relationship between observation signal power calculated from the observation signal and error signal power calculated from the error signal is further included. As a calculation method of signal power, it is sufficient that an absolute value of a signal value, a square value, or a power total value in a preset bandwidth on a high-frequency spectrogram, or the like is used. It is possible to ensure signal quality of biological information on the basis of the output signal quality calculation unit. Therefore, highly-accurate biological information can be obtained

It is desirable that, in the first embodiment, a postprocessing filter unit that further reduces residual noise included in the error signal, by low pass filter processing is further included. Therefore, highly-accurate biological information can be obtained

It is desirable that, in the first embodiment, the active state analysis unit 162 that analyzes an active state on the basis of the observation signal, the body motion signal and/or the pressure signal, and determines a reference signal from the body motion signal or the pressure signal on the basis of the analysis result is further included.

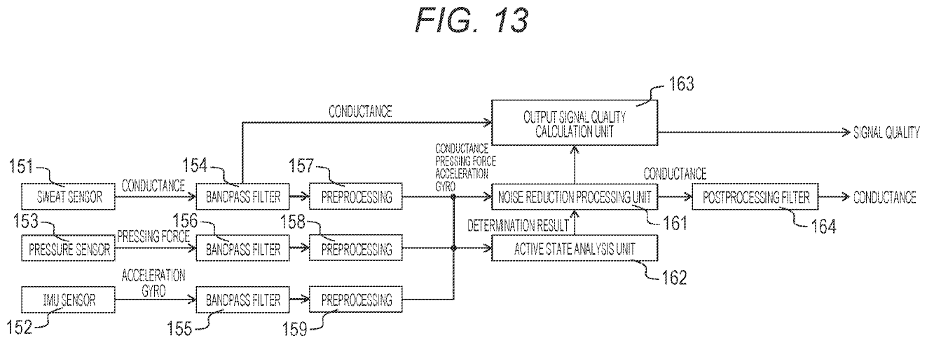

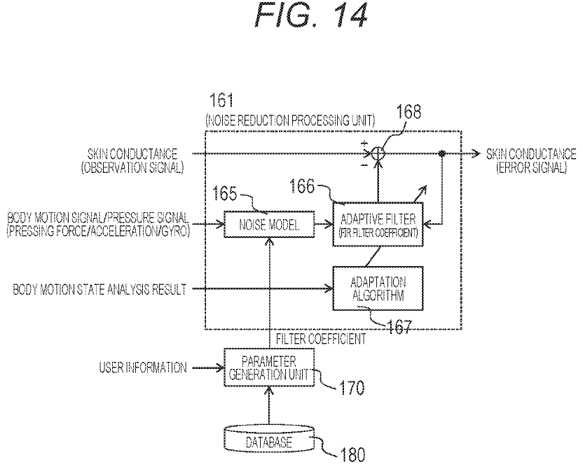

An operation of the active state analysis unit 162 will be described in more detail with reference to FIGS. 10 to 13. The active state analysis unit 162 may be any of the first activity analysis unit (refer to FIG. 10), the second activity analysis unit (refer to FIG. 11), or the third activity analysis unit (refer to FIG. 12). The active state analysis unit 162 will be described using these examples, but the active state analysis unit 162 is not limited to these examples. The redundant description of the similar configurations will be appropriately omitted.

<4-1. First Active State Analysis Unit>

Referring to FIG. 10, the first active state analysis unit includes the first sensor analysis unit 61 that determines a non-attached state or a noncontact state, and the second sensor analysis unit 62 that determines an active state. The first active state analysis unit is configured to receive signals input from the first sensor unit 151 and the second sensor unit 152, and may be further configured to further receive a signal input from the third sensor unit 153.

Then, the first active state analysis unit is configured to determine that the sensor is not attached or not in contact, in a case where it is determined in the first sensor analysis unit 61 that an observation signal is smaller than a threshold value. In a case where it is determined in the first sensor analysis unit 61 that an observation signal is equal to or larger than a threshold value, the first active state analysis unit shifts determination to the second sensor analysis unit 62. The first active state analysis unit is configured to output, in a case where it is determined in the second sensor analysis unit 62 that a body motion signal is equal to or larger than a threshold value, the body motion signal to the noise reduction processing unit 161 as a reference signal. In a case where it is determined in the second sensor analysis unit 62 that a body motion signal is smaller than a threshold value, the first active state analysis unit determines a biological body is in a rest state.

In a case where it is determined that biological body is in an active state, using a body motion signal having passed through the bandpass filter unit 155, as a reference signal, the noise reduction processing unit 161 obtains an error signal by subtracting the reference signal from an observation signal. In a case where it is determined that biological body is in a quasi-rest state, without using a body motion signal as a reference signal, an instruction to use an observation signal as-is is issued to the noise reduction processing unit 161. Furthermore, an observation signal subjected to BPF processing is output to the output signal quality calculation unit 163, and signal quality is calculated. Therefore, biological information can be obtained more accurately.

<Operation of First Active State Analysis Unit>

The first active state analysis unit causes the first sensor analysis unit 61 to determine a contact state of a biological sensor (Step 1). The first sensor analysis unit 61 determines whether or not an observation signal input from the sweat sensor 151 is equal to or larger than a threshold value (Step 2). In a case where it is determined that the observation signal is smaller than a threshold value, it is determined that the sensor is not attached/not in contact, and the first active state analysis unit notifies the user of this (image display, speech display, and the like). In a case where the observation signal is equal to or larger than a threshold value, it is determined that biological sensor contact is good, and the first active state analysis unit causes the body motion analysis unit 62 to determine whether or not the user is in an active state.

The second sensor analysis unit 62 processes a body motion signal input from the IMU sensor 152, and determines whether or not the processed signal is equal to or larger than a threshold value (Step 3). In a case where the processed signal is equal to or larger than a threshold value, it is determined that the user is in an active state, and the first active state analysis unit transmits a body motion signal to the noise reduction processing unit 161 as a reference signal. In a case where the processed signal is smaller than a threshold value, it is determined that the user is in a rest state, and the first active state analysis unit transmits nonexistence of body motion noise to the noise reduction processing unit 161.

In a case where the noise reduction processing unit 161 uses a body motion signal as a reference signal on the basis of an analysis result of the first active state analysis unit, the body motion signal is regarded as body motion noise (Step 4). Then, the noise reduction processing unit 161 calculates an error signal obtained by subtracting body motion noise included in an observation signal, and outputs the error signal as biological information. In a case where it is determined that the user is in a rest state and body motion noise is not included, on the basis of an analysis result of the first active state analysis unit, the noise reduction processing unit 161 determines to use an observation signal as-is, and notifies the output signal quality calculation unit of the observation signal (Step 5). Biological information is output from the output signal quality calculation unit 163 to which the observation signal is input.

Note that the biological information processing apparatus including the first active state analysis unit may include the first sensor unit 151 and the second sensor unit 152, and may further include the third sensor unit 153.

<4-2. Second Active State Analysis Unit>

Referring to FIG. 11, the second active state analysis unit includes the above-described first sensor analysis unit 61 and the third sensor analysis unit 63 that determines a quasi-rest state. The second active state analysis unit is configured to receive signals input from the first sensor unit 151 and the third sensor unit 153, and may be further configured to further receive a signal input from the second sensor unit 152.

Then, the second active state analysis unit is configured to determine that the sensor is not attached or not in contact, in a case where it is determined in the first sensor analysis unit 61 that an observation signal is smaller than a threshold value. In a case where it is determined in the first sensor analysis unit 61 that an observation signal is equal to or larger than a threshold value, the second active state analysis unit determines that the user is in an inactive state, and shifts determination to the third sensor analysis unit 63. The second active state analysis unit is configured to determine, in a case where it is determined in the third sensor analysis unit 63 that a pressure signal is equal to or larger than a threshold value, that the user is in a quasi-rest state, and output the pressure signal to the noise reduction processing unit 161. In a case where it is determined in the third sensor analysis unit 63 that a pressure signal is smaller than a threshold value, the second active state analysis unit determines that the user is in a rest state, and outputs an observation signal as-is to the noise reduction processing unit 161 without a reference signal. Furthermore, when the second active state analysis unit outputs nonexistence of a reference signal, the second active state analysis unit can also transmit nonexistence of a reference signal to the output signal quality calculation unit 163, and signal quality is transmitted from the output signal calculation unit 163.

<Operation of Second Active State Analysis Unit>

The second activity analysis unit causes the first sensor analysis unit 61 to determine a contact state of a biological sensor (Step 1). The first sensor analysis unit 61 determines whether or not an observation signal input from the sweat sensor 151 is equal to or larger than a threshold value (Step 2). In a case where it is determined that the observation signal is smaller than a threshold value, it is determined that the sensor is not attached/not in contact, and the second active state analysis unit notifies the user of this (image display, speech display, and the like). In a case where the observation signal is equal to or larger than a threshold value, it is determined that biological sensor contact is good, and in a case where an inactive state is further set, the second active state analysis unit causes the third sensor analysis unit 63 to determine whether or not the user is in a rest state.

The third sensor analysis unit 63 processes a pressure signal input from the pressure sensor 153, and determines whether or not the processed signal is equal to or larger than a threshold value (Step 3). In a case where the processed signal is equal to or larger than a threshold value, it is determined that the user is in a quasi-rest state, and the second active state analysis unit transmits a pressure signal to the noise reduction processing unit 161 as a reference signal. In a case where the processed signal is smaller than a threshold value, it is determined that the user is in a rest state, and the second active state analysis unit transmits nonexistence of body motion noise to the noise reduction processing unit 161.

In a case where the noise reduction processing unit 161 uses a pressure signal as a reference signal on the basis of an analysis result of the second active state analysis unit, the pressure signal is regarded as body motion noise (Step 4). Then, the noise reduction processing unit 161 calculates an error signal obtained by subtracting body motion noise included in an observation signal, and outputs the error signal as biological information. In a case where body motion noise is not included, the noise reduction processing unit 161 determines to use an observation signal as-is, and notifies the output signal quality calculation unit of the observation signal (Step 5). Biological information is output from the output signal quality calculation unit 163 to which the observation signal is input.

Note that the biological information processing apparatus including the second active state analysis unit may include the first sensor unit 151 and the third sensor unit 153, and may further include the second sensor unit 152.

<4-3. Third Active State Analysis Unit>

Referring to FIG. 12, the third active state analysis unit includes the first sensor analysis unit 61, the second sensor analysis unit 62, and the third sensor analysis unit 63 as described above. The third active state analysis unit is configured to receive signals input from the first sensor unit 151, the second sensor unit 152, and the third sensor unit 153.

Then, the third active state analysis unit determines that the sensor is not attached or not in contact, in a case where it is determined in the first sensor analysis unit 61 that an observation signal is smaller than a threshold value. In a case where it is determined in the first sensor analysis unit 61 that an observation signal is equal to or larger than a threshold value, the third active state analysis unit shifts determination to the second sensor analysis unit 62. In a case where it is determined in the second sensor analysis unit 62 that a body motion signal is equal to or larger than a threshold value, the third active state analysis unit outputs the body motion signal to the noise reduction processing unit 161 as a reference signal. In a case where it is determined in the second sensor analysis unit 62 that a body motion signal is smaller than a threshold value, the third active state analysis unit shifts determination to the third sensor analysis unit 63. After the shift, the third active state analysis unit determines, in the third sensor analysis unit 63, that the user is in a quasi-rest state or a rest state. In a case where it is determined in the third sensor analysis unit 63 that a pressure signal is equal to or larger than a threshold value, the third active state analysis unit determines that the user is in a quasi-rest state, and outputs the pressure signal to the noise reduction processing unit 161. In a case where it is determined in the third sensor analysis unit 63 that a pressure signal is smaller than a threshold value, the third active state analysis unit determines that the user is in a rest state, and outputs an observation signal as-is to the noise reduction processing unit 161 without a reference signal. Furthermore, when the third active state analysis unit outputs nonexistence of a reference signal, the third active state analysis unit can also transmit nonexistence of a reference signal to the output signal quality calculation unit 163, and signal quality is transmitted from the output signal calculation unit 163.

<Operation of Third Active State Analysis Unit>

The third active state analysis unit causes the first sensor analysis unit 61 to determine a contact state of a biological sensor (Step 1). The first sensor analysis unit 61 determines whether or not an observation signal input from the sweat sensor 151 is equal to or larger than a threshold value (Step 2). In a case where it is determined that the observation signal is smaller than a threshold value, it is determined that the sensor is not attached/not in contact, and the third active state analysis unit notifies the user of this (image display, speech display, and the like). In a case where the observation signal is equal to or larger than a threshold value, it is determined that biological sensor contact is good, and the third active state analysis unit causes the second sensor analysis unit 62 to determine whether or not the user is in an active state.

The second sensor analysis unit 62 processes a body motion signal input from the IMU sensor 152, and determines whether or not the processed signal is equal to or larger than a threshold value (Step 3). In a case where the processed signal is equal to or larger than a threshold value, it is determined that the user is in an active state, and the third active state analysis unit transmits a body motion signal to the noise reduction processing unit 161 as a reference signal. In a case where the processed signal is smaller than a threshold value, the third active state analysis unit causes the third sensor analysis unit 63 to determine whether or not the user is in a rest state.

The third sensor analysis unit 63 processes a pressure signal input from the pressure sensor 153, and determines whether or not the processed signal is equal to or larger than a threshold value (Step 4). In a case where the processed signal is equal to or larger than a threshold value, it is determined that the user is in a quasi-rest state, and the third active state analysis unit transmits a pressure signal to the noise reduction processing unit 161 as a reference signal. In a case where the processed signal is smaller than a threshold value, it is determined that the user is in a rest state, and the third active state analysis unit transmits nonexistence of body motion noise to the noise reduction processing unit 161.

In a case where the noise reduction processing unit 161 uses a body motion signal as a reference signal on the basis of an analysis result of the third active state analysis unit, the body motion signal is regarded as body motion noise, or in a case where a pressure signal is used as a reference signal, the pressure signal is regarded as body motion noise (Step 5). Then, the noise reduction processing unit calculates an error signal obtained by subtracting body motion noise included in an observation signal, and outputs the error signal as biological information. In a case where body motion noise is not included, the noise reduction processing unit 161 determines to use an observation signal as-is, and notifies the output signal quality calculation unit of the observation signal (Step 6). Biological information is output from the output signal quality calculation unit 163 to which the observation signal is input.

Note that the biological information processing apparatus including the third active state analysis unit may include the first sensor unit 151, the second sensor unit 152, and the third sensor unit 153.

<Operation of Biological Information Processing Apparatus According to First Embodiment>

An example of an operation in the biological information processing apparatus according to the first embodiment will be described below, but the operation is not limited to this. Noise reduction processing of biological information can be thereby performed.

Fluctuation components are extracted using a bandpass filter from a signal measured by each sensor. Active state analysis is performed from skin conductance, an acceleration signal, and a pressure signal. A flow of the active state analysis will be described with reference to FIGS. 9 and 12, but the flow is not limited to this.

First of all, a contact state between an electrode pair and the skin is determined from skin conductance using a threshold value. For example, if skin conductance is equal to or larger than a threshold value, it is determined that a wristband type sweat sensor device is in contact with a measurement region. It skin conductance is equal to or smaller than a threshold value, it is determined that a wristband type sweat sensor device is not in contact, and determines that the device is not attached/not in contact. Next, whether or not the user is in an active state is determined by threshold value determination of an output result of the first sensor analysis unit. In the active state analysis unit, an active state is calculated from a body motion signal.

In a case where an acceleration sensor is a triaxial acceleration sensor, a value of a maximum value filter is output by buffering a norm value of a body motion signal. If a value is equal to or larger than a threshold value, it is determined that the user is in an active state.

Lastly, a pressing force state is determined by threshold value determination of an output result of the third sensor analysis unit 63. The third sensor analysis unit 63 calculates a temporal pressing force change between the electrode pair and the skin. A value of a maximum value filter is output by buffering a differential absolute value of a pressure signal. If a value is equal to or larger than a threshold value, it is determined that pressing force changes. In this case, it is determined that the user is in a quasi-rest state.

Using an adaptive filter while regarding skin conductance as an observation signal, and an acceleration signal and a pressure signal as a reference signal, an error signal (skin conductance) from which body motion noise superimposed on the skin conductance is reduced is calculated.

In the case of this first embodiment, in the state of the active state analysis unit 162 of the above-described step, a reference signal is selected and used. For example, in a case where it is determined that the user is in an active state, noise removal is performed using an adaptive filter while regarding triaxial acceleration as a reference signal. In a case where it is determined that the user is in a quasi-rest state, it is only required that a plurality of (for example, eight) pressing force changes is regarded as a reference signal, and noise removal is performed using an adaptive filter.

Moreover, for determining whether or not noise is reduced by adaptive filter processing, the output signal quality calculation unit 163 determines whether or not error signal power becomes smaller than observation signal power. As an example of a calculation method of signal power, it is sufficient that an absolute value of a signal value, a square value, or a power total value in a preset bandwidth on a high-frequency spectrogram, or the like is used.

Moreover, the postprocessing filter unit can perform low pass filter processing for removing residual noise included in an output signal (error signal) of adaptive filter processing.

5. Biological Information Processing Apparatus According to Second Embodiment

The redundant description of the configurations similar to those in the first embodiment will be omitted. An information processing apparatus according to the second embodiment of the present technology further includes a preprocessing unit that preprocesses a signal to be input to the noise reduction processing unit 161. The preprocessing unit that performs absolute value processing of a signal on fluctuation components subjected to bandpass filter processing, as preprocessing of a signal to be input to the noise reduction processing unit 161 is further included. It is preferable that preprocessing units 157, 158, and 159 are respectively provided after BPF units 154, 155, and 156. With this configuration, it becomes possible to effectively remove noise from harmonic components of body motion noise frequency. In contrast to the above-described first embodiment, by performing absolute value processing on fluctuation components of the measured high-voltage signal that have been subjected to BPF processing, the frequency of the signal is made higher, and can be used as a reference signal of an adaptive filter processing unit. With this configuration, harmonic components of body motion noise can be dealt with and a noise reduce effect therefore improves. Therefore, biological information can be obtained more accurately. Note that, in a case where the first sensor analysis unit 61 is a sweat sensor, the first sensor analysis unit 61 is preferably a contact analysis unit 61. Furthermore, in a case where the second sensor analysis unit 62 is an acceleration sensor, the second sensor analysis unit 62 is preferably a body motion analysis unit 62. Furthermore, in a case where the third sensor analysis unit 63 is a pressure sensor, the third sensor analysis unit 63 is preferably a pressing force analysis unit 63.

<Operation of Biological Information Processing Apparatus According to Second Embodiment>

An example of an operation in the biological information processing apparatus according to the second embodiment will be described below, but the operation is not limited to this. Noise reduction processing of biological information can be thereby performed. The biological information processing apparatus of the second embodiment adds the configuration of the above-described first embodiment. With this configuration, absolute value processing of a signal is performed as preprocessing on fluctuation components subjected to bandpass filter processing, and the frequency of a reference signal is easily made higher (doubled). With this configuration, it becomes possible to effectively remove noise from harmonic components of body motion noise frequency.

The observation signal, the body motion signal, and the pressure signal processed by the preprocessing unit 157, the preprocessing unit 158, and the preprocessing unit 159 of the second embodiment are appropriately output to an active state analysis unit in the biological information processing apparatus of the embodiment. On the basis of these observation signal and body motion signal, the first active state analysis unit performs <operation of first active state analysis unit> described above. Furthermore, on the basis of these observation signal and pressure signal, the second active state analysis unit performs <operation of second active state analysis unit> described above. Furthermore, on the basis of these observation signal, body motion signal, and pressure signal, <operation of third active state analysis unit> described above is performed.

6. Biological Information Processing Apparatus According to Third Embodiment

The redundant description of the configurations similar to those in the first or second embodiment will be omitted. A biological information processing apparatus according to the third embodiment of the present technology includes the noise reduction processing unit 161, and the noise reduction processing unit 161 further includes an adaptive filter processing unit 166 (refer to FIG. 14). The noise reduction processing unit 161 is configured to calculate an error signal obtained by subtracting a reference signal of the adaptive filter processing unit 166 as body motion noise from an observation signal. Note that, in a case where the first sensor analysis unit 61 is a sweat sensor, the first sensor analysis unit 61 is preferably a contact analysis unit 61. Furthermore, in a case where the second sensor analysis unit 62 is an acceleration sensor, the second sensor analysis unit 62 is preferably a body motion analysis unit 62. Furthermore, in a case where the third sensor analysis unit 63 is a pressure sensor, the third sensor analysis unit 63 is preferably a pressing force analysis unit 63.

The biological information processing apparatus according to the third embodiment may further include a parameter generation unit 170, and may further include a database 180 on the outside or inside of the apparatus in such a manner that transmission and reception can be performed with the parameter generation unit 170. The parameter generation unit 170 is configured to acquire parameter information accumulated in the database 180 on the basis of skin conductance information obtained immediately after the biological information processing apparatus is attached. Moreover, the parameter generation unit 170 is configured to generate a transfer function (filter coefficient) to that is based on a pressing force change of the user, from the acquired parameter information.

It is preferable that the noise reduction processing unit 161 includes the adaptive filter processing unit 166 and a subtractor 168. Furthermore, it is desirable that the noise reduction processing unit 161 includes a unit 167 that can store a noise model (transfer function) and an adaptation algorithm. The adaptive filter processing unit 166 is configured to calculate a reference signal value obtained by further convoluting a transfer function into an input reference signal, and output the value. It is preferable that the adaptive filter processing unit 166 is configured to appropriately receive an adaptive filter coefficient for update that is input from an adaptation algorithm, and preliminarily correct an input noise model (transfer function).

The noise reduction processing unit 161 includes the subtractor 168 that calculates an error signal obtained by subtracting the reference signal value output from the adaptive filter processing unit 166, from an observation signal, and is configured to output, by the subtractor, the error signal as corrected skin conductance.

In noise reduction processing, it is possible to use fluctuation components subjected to BPF processing, as a reference signal of an adaptive filter as-is. In a case where noise removal is performed using an adaptive filter, it is desirable that the reference signal has high correlation with noise included in an observation signal. It is therefore more preferable that a transfer coefficient preliminarily calculated considering a body motion noise factor (pressing force change, body motion change) attributed to a body motion is obtained in advance, and the transfer coefficient is used as an adaptive filter.

It is preferable that the transfer coefficient is preinstalled as an adaptive filter as a default setting before an individual user acquires biological information. Moreover, by an individual user acquiring biological information, the adaptive filter can be appropriately updated using an adaptation algorithm. By updating an adaptation algorithm, it is possible to detect body motion noise generated in accordance with the characteristic (movement of a body and the like) of an individual user. Therefore, body motion noise included in an observation signal of biological information can be accurately reduced in accordance with an individual user.

In the third embodiment, the following description will be given of an example case where noise reduction processing is performed using a signal convoluted into fluctuation components of a pressing force change, as a reference signal of an adaptive filter, but the present technology is not limited to this.

The noise reduction processing unit 161 is configured to receive a model coefficient (filter coefficient) calculated by measuring a body motion noise factor, and a signal (specifically, body motion signal, pressing force signal) input from each sensor. Furthermore, the noise reduction processing unit may be configured to calculate a model coefficient from a signal input from each sensor.

In the third embodiment, an adaptation algorithm of an adaptive filter is not specifically limited, but the description will be given with reference to an NLMS algorithm as an example.

In the NLMS algorithm, an adaptive filter coefficient w (Formula (1)) of an adaptive filter is updated using an update formula of the following formula (2). Note that, in the present embodiment, as the adaptive filter coefficient w, an FIR filter coefficient calculated in advance is used as described later. In the formula, n denotes a sample number. w(n+1) represents an updated adaptive filter coefficient.

Here, p is a positive constant for determining an update amount of the adaptive filter coefficient w, and is called a step size. In the case of the present embodiment, on the basis of an active state analysis result, by setting a step size to a larger size than a normal size within a preset time period from when a rapid change of an active state is detected, a convergence time is improved. For example, a step size within a certain time period is set to M times. In the present embodiment, the description has been given using an example of the NLMS algorithm, but another adaptation algorithm can be similarly adapted.

$$\begin{matrix}[ Math . 1 ] \\ & \\ e ( n ) = d - w ( n ) \cdot {x}^{T} ( n ) & ( 1 ) \\ w ( n + 1 ) = w ( n ) + μ \frac{e ( n ) \cdot x ( n )}{{\sum }_{i = 0}^{x - 1} {x}^{2} ( n - i )} & ( 2 )\end{matrix}$$

In a case where a pressure sensor is built into a band, because a pressing force change between an electrode and the skin is not measured directly, a pressing force change convoluting a transfer function that is based on the characteristic of elastic material (for example, band material, material of deformable member, and the like) is applied to a pressing force sensor. In such a case, because a transfer coefficient is included in a pressing force signal, it is desirable to add the transfer coefficient to a reference signal. By adaptive filter processing in which a transfer coefficient is preinstalled before acquisition of biological information, it is possible to reduce noise applied to a pressure sensor by elastic material, from an observation signal.

Another pressure sensor is disposed on the surface of an electrode (electrode on the side contacting the skin), and a pressing force change Pi in the band (electrode on the side contacting the band) when impulsive pressing force change Po is applied to the surface is measured. By performing system identification from the measured Pi and Po, a filter coefficient is estimated assuming that a transfer function H of the band is a finite impulse response (FIR) filter type.

Using the FIR filter coefficient estimated as described above, a signal obtained by convoluting the coefficient into fluctuation components of a pressing force change that have been subjected to BPF processing is regarded as a reference signal of an adaptive filter. Using the reference signal of the adaptive filter, an error signal obtained by subtracting body motion noise included in an observation signal is calculated.

<Operation of Biological Information Processing Apparatus According to Third Embodiment>

An example of an operation in the biological information processing apparatus according to the third embodiment will be described below, but the operation is not limited to this. Noise reduction processing of biological information can be thereby performed. The biological information processing apparatus of the third embodiment adds the configuration of the above-described first or first embodiment.

The observation signal, the body motion signal, and the pressure signal that have been processed in the above-described first or second embodiment are appropriately output to an active state analysis unit in the biological information processing apparatus according to the third embodiment. An operation of the active state analysis unit that is performed at this time is as described above in <Operation of Biological Information Processing Apparatus According to First Embodiment> or <Operation of Biological Information Processing Apparatus According to Second Embodiment>. By the active state analysis unit according to the third embodiment, not attached/not in contact, an active state, a quasi-rest state, or a rest state is determined. A body motion signal or a pressure signal is thereby output from the active state analysis unit to a noise reduction processing unit as a reference signal.

The noise reduction processing unit reads a noise model (transfer function) and an adaptation algorithm, and outputs the noise model and the adaptation algorithm to an adaptive filter processing unit. The noise processing reduction unit outputs the determined reference signal or non-existence of body motion noise to the adaptive filter processing unit on the basis of a result of the above-described active state analysis unit.

The adaptive filter processing unit calculates a reference signal value by further adding a transfer function to the reference signal input from a sensor or the non-existence of a reference signal, and convoluting the transfer function, and outputs the value. An error signal is obtained by subtracting the reference signal value subjected to adaptive filter processing, from an observation signal.

Moreover, the adaptive filter processing unit corrects a noise model (transfer function) input in advance, by an adaptive filter coefficient for update being appropriately input from an adaptation algorithm.

7. Configuration Example of Analysis Apparatus

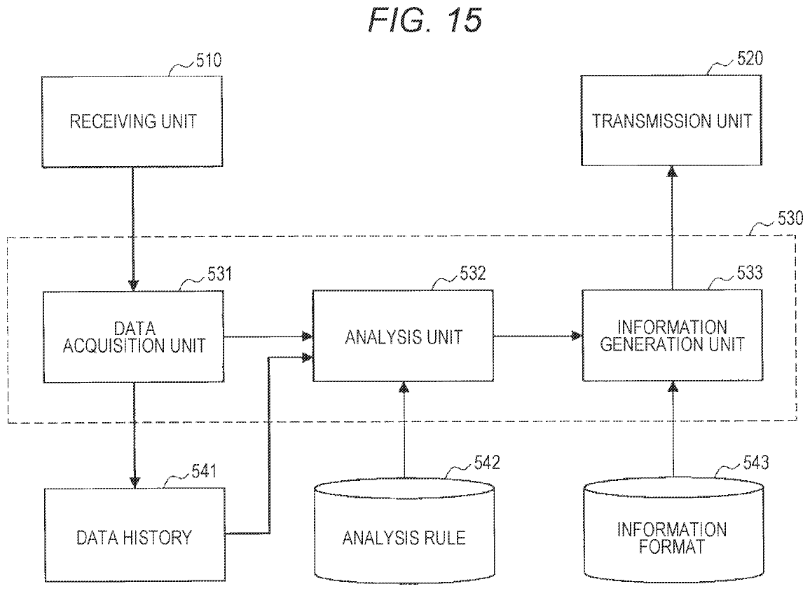

FIG. 15 is a block diagram illustrating a schematic configuration example of a biological information analysis apparatus according to an embodiment of the present disclosure.

The biological information analysis apparatus is an apparatus that executes analysis that is based on skin conductance measured in the sensor apparatus 100, and is implemented as the server 300, the terminal apparatus 400, or the sensor apparatus 100. In the example illustrated in FIG. 15, the analysis apparatus includes a receiving unit 510, a transmission unit 520, and a processing unit 530. The receiving unit 510 and the transmission unit 520 are implemented by various communication devices that communicate via the network 200 and the like, for example. Furthermore, the processing unit 530 is implemented by a processor such as a central processing unit (CPU) operating in accordance with a program stored in a memory or a storage. As necessary, the processing unit 530 refers to a data history 541, an analysis rule 542, and/or an information format 543 stored in a memory or a storage. Regarding each configuration, for example, Japanese Patent Application Laid-Open 2016-97159 can be referred to.

The receiving unit 510 receives data of skin conductance measured in the sensor apparatus 100. For example, in a case where the analysis apparatus is implemented as the server 300, the receiving unit 510 receives data from the sensor apparatus 100 via the network 200. Furthermore, in a case where the analysis apparatus is implemented as the terminal apparatus 400, the receiving unit 510 receives data from the sensor apparatus 100 via the network 200 or directly via Bluetooth (registered trademark) or the like. Alternatively, in a case where the analysis apparatus is implemented as the sensor apparatus 100, the receiving unit 510 internally receives data via a bus or the like.

The transmission unit 520 transmits information that is based on a result of analysis executed on the basis of skin conductance. For example, in a case where the analysis apparatus is executed as the server 300 and information is output by the sensor apparatus 100 using a display 110 or the like, the transmission unit 520 transmits information to the sensor apparatus 100 via the network 200. Furthermore, in a case where the analysis apparatus is implemented as the server 300 and information is output by the terminal apparatus 400 using a display 410 or the like, the transmission unit 520 transmits information to the terminal apparatus 400 via the network 200.

On the other hand, in a case where the analysis apparatus is implemented as the terminal apparatus 400, and information is output by the sensor apparatus 100 using the display 110 or the like, the transmission unit 520 transmits information to the sensor apparatus 100 via the network 200 or directly via Bluetooth (registered trademark) or the like. In a case where the analysis apparatus is implemented as the terminal apparatus 400, and information is output by the terminal apparatus 400 using the display 410 or the like, the transmission unit 520 internally transmits information via a bus or the like. In a case where the analysis apparatus is implemented as the sensor apparatus 100, and information is output by the sensor apparatus 100 using the display 110 or the like, similarly, the transmission unit 520 internally transmits information via a bus or the like. In a case where the analysis apparatus is implemented as the sensor apparatus 100, and information is output by the terminal apparatus 400 using the display 410 or the like, the transmission unit 520 transmits information the terminal apparatus 400 via the network 200 or directly via Bluetooth (registered trademark) or the like.

In the processing unit 530, a data acquisition unit 531 acquires data received by the receiving unit 510. As described above, the acquired data includes data of skin conductance measured by an electrode pair that contacts the skin of the user in the sensor apparatus 100. The data acquisition unit 531 may provide the acquired data to an analysis unit 532 and accumulate the data into the data history 541.

The analysis unit 532 extracts biological information of the user from data provided by the data acquisition unit 531. Here, the biological information includes EDA, for example. As described above, in the sensor apparatus 100, the above-described noise-reduced skin conductance may be calculated Moreover, the analysis unit 532 may convert extracted biological information such as EDA, into another type of biological information such as an activity level of a sympathetic nerve or a parasympathetic nerve. In executing such analysis, the analysis unit 532 may refer to the preset analysis rule 542. Furthermore, for executing analysis that is based on latest data, the analysis unit 532 may refer to the past data history 541.

An information generation unit 533 generates information to be provided to the user, on the basis of a result of analysis executed by the analysis unit 532. The biological information such as EDA that is extracted by the analysis unit 532 from skin conductance can be used for various purposes. For example, the biological information can be used for detecting feeling of the user such as strain, relax, joy, and sadness. Information regarding the detected feeling may be referred to by the user, or may be referred to by another user. The detected feeling can be effectively used as a communication tool in a situation in which facial expression or the like of the other party cannot be directly seen, as in a case where a plurality of users watches a shared moving image, for example. Furthermore, biological information may be evaluated on the basis of a relationship with the activity of the user. For example, from biological information obtained when the user is playing golf, a mental state of the user during the play may be estimated. Furthermore, for example, from biological information obtained when the user is doing yoga, whether or not yoga contributes to the improvement of a mental state of the user may be estimated. The information generation unit 533 generates information that is based on biological information, in accordance with the information format 543 prepared in advance.

In the present embodiment, by the above-described functional configuration, for example, influence attributed to a body motion for which body motion noise is included in an observation signal of skin conductance, for example, can be removed, and an autonomic nerve activity or a metabolite level of the user can be accurately estimated. Furthermore, for example, in a case where the sensor apparatus 100 or the terminal apparatus 400 includes a sensor such as a dermotherm and an acceleration meter other than the electrode pair, using data provided by these sensors, together with EDA, a change in EDA that is attributed to temperature, diet, exercise, and the like can be identified. Note that a plurality of regions having different changes in conductance obtained by EDA is not limited to the inside and outside of a wrist, and can be the inside and outside of a finger, the inside and outside of an upper arm, the inside and outside of a neck, or the like. The sensor apparatus 100 is not limited to a wrist-wear, and may have a shape attachable to these regions, for example.

8. Hardware Configuration

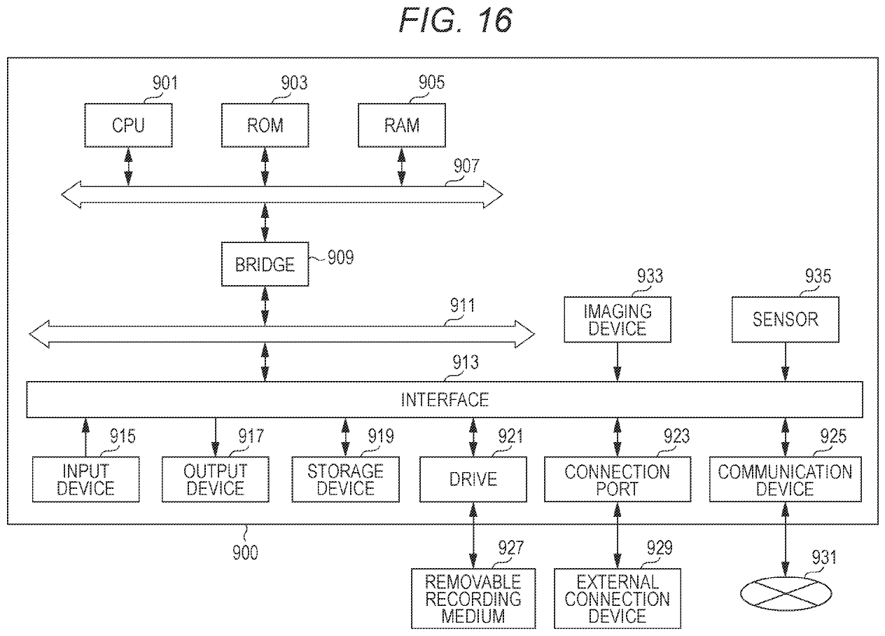

A hardware configuration of the information processing device according to an embodiment of the present disclosure will be described with reference to FIG. 16. FIG. 16 is a block diagram illustrating a hardware configuration example of the information processing apparatus according to an embodiment of the present disclosure. An information processing apparatus 900 illustrated in the drawing can implement an analysis apparatus in the above-described embodiment, for example. More specifically, the analysis apparatus can be the server 300, the terminal apparatus 400, or se the sensor apparatus 100.

The information processing apparatus 900 includes a central processing unit (CPU) 901, a read only memory (ROM) 903, and a random access memory (RAM) 905. Furthermore, the information processing apparatus 900 may include a host bus 907, a bridge 909, an external bus 911, an interface 913, an input device 915, an output device 917, a storage device 919, a drive 921, a connection port 923, and a communication device 925. Moreover, the information processing apparatus 900 may include, as necessary, an imaging device 933 and a sensor 935. The information processing apparatus 900 may include, in place of the CPU 901 or together with the CPU 901, a processing circuit such as a digital signal processor (DSP), an application specific integrated circuit (ASIC), or a field-programmable gate array (FPGA).

The CPU 901 functions as an arithmetic processing unit and a control device, and controls all or a part of the operations in the information processing apparatus 900 in accordance with various programs recorded in the ROM 903, the RAM 905, the storage device 919, or a removable recording medium 927. The ROM 903 stores programs, calculation parameters, or the like that are to be used in the CPU 901. The RAM 905 temporarily stores programs used in the execution of the CPU 901, parameters appropriately changing in the execution, and the like. The CPU 901, the ROM 903, and the RAM 905 are connected to each other by the host bus 907 including an internal bus such as a CPU bus. Moreover, the host bus 907 is connected, via the bridge 909, to the external bus 911 such as a peripheral component interconnect/interface (PCI) bus.

The input device 915 is a device to be operated by the user, such as, for example, a mouse, a keyboard, a touch panel, a button, a switch, and a lever. The input device 915 may be a remote-control device that uses infrared rays or other radiowaves, for example, or may be an external connection device 929 such as a mobile phone that supports operations of the information processing apparatus 900. The input device 915 includes an input control circuit that generates an input signal on the basis of information input by the user, and outputs the input signal to the CPU 901. By operating the input device 915, the user inputs various types of data to the information processing apparatus 900 or instructs the information processing apparatus 900 to perform a processing operation.

The output device 917 includes a device that can notify the user of acquired information using a sense such as a visual sense, an auditory sense, and a tactile sense. The output device 917 can be, for example, a display device such as a liquid crystal display (LCD) or an organic Electro-Luminescence (EL) display, an audio output device such as a speaker or headphones, a vibrator, or the like. The output device 917 outputs a result obtained by processing of the information processing apparatus 900, as a video including a text, an image, or the like, sound such as voice or audio, vibration, or the like.

The storage device 919 is a device for data storage that is formed as an example of a storage unit of the information processing apparatus 900. The storage device 919 includes, for example, a magnetic storage unit device such as a hard disk drive (HDD), a semiconductor storage device, an optical storage device, a magnetooptical storage device, or the like. The storage device 919 stores, for example, programs executed by the CPU 901, various types of data, various types of data acquired from the outside, and the like.

The drive 921 is a reader/writer for the removable recording medium 927 such as a magnetic disc, an optical disk, a magnetooptical disk, or a semiconductor memory, and is built into the information processing apparatus 900 or externally attached thereto. The drive 921 reads out information recorded in the attached removable recording medium 927, and outputs the information to the RAM 905. Furthermore, the drive 921 writes records into the attached removable recording medium 927.

The connection port 923 is a port for connecting a device to the information processing apparatus 900. For example, the connection port 923 can be a universal serial bus (USB) port, an IEEE1394 port, a small computer system interface (SCSI) port, or the like. Furthermore, the connection port 923 may be an RS-232C port, an optical audio terminal, a high-definition multimedia interface (HDMI) (registered trademark) port, or the like. By connecting the external connection device 929 to the connection port 923, various types of data can be replaced between the information processing apparatus 900 and the external connection device 929.

The communication device 925 is a communication interface including a communication device or the like for connecting to a communication network 931, for example. The communication device 925 can be, for example, a local area network (LAN), Bluetooth (registered trademark), Wi-Fi, a communication card for a wireless USB (WUSB), or the like. Furthermore, the communication device 925 may be a router for optical communication, a router for Asymmetric Digital Subscriber Line (ADSL), various communication modems, or the like. The communication device 925 transmits and receives a signal or the like using a predetermined protocol such as TCP/IP, with the Internet or another communication device, for example. Furthermore, the communication network 931 connected to the communication device 925 is a network connected in a wired or wireless manner, and can include, for example, the Internet, home LAN, infrared communication, radiofrequency communication, satellite communications, and the like.

The imaging device 933 is a device that generates a captured image by capturing an image of a real space using various members such as an image sensor such as a complementary metal oxide semiconductor (CMOS) image sensor or a charge coupled device (CCD) image sensor, for example, and a lens for controlling formation of a subject image onto the image sensor. The imaging device 933 may be a device that captures a still image or may be a device that captures a moving image.

The sensor 935 is various sensors such as, for example, an acceleration sensor, a pressure sensor, an angular velocity sensor, a geomagnetic sensor, an illumination sensor, a temperature sensor, a barometer, sound sensor (microphone), or the like. The sensor 935 acquires information regarding the state of the information processing apparatus 900 such as, for example, the orientation of the casing of the information processing apparatus 900, and information regarding a surrounding environment of the information processing apparatus 900 such as brightness or noise around the information processing apparatus 900. Furthermore, the sensor 935 may include a global positioning system (GPS) receiver that receives a GPS signal and measures latitude, longitude, and altitude of the apparatus.

Heretofore, an example of the hardware configuration of the information processing apparatus 900 has been described. Each of the above-described components may be formed using a general-purpose member, or may be formed by hardware dedicated to the function of each component. The configuration can be appropriately changed in accordance with the technical level at the implementation timing.

Note that the present technology can also employ the following configurations.

[1] A biological information processing apparatus including:

a noise reduction processing unit configured to calculate an error signal obtained by subtracting body motion noise included in an observation signal from a first sensor unit configured to measure biological body affect as an observation signal, on the basis of a body motion signal from a second sensor unit configured to measure a body motion change, and/or a pressure signal from a third sensor unit configured to measure a pressing force change in skin.

[2] The biological information processing apparatus according to [1] described above, in which the first sensor is a sweat sensor unit.
[3] The biological processing information apparatus according to [1] or [2] described above, in which the noise reduction processing unit is configured to regard either the body motion signal or the pressure signal as a reference signal, and calculate an error signal by subtracting body motion noise from the observation signal, using the reference signal.
[4] The biological information processing apparatus according to any one of [1] to [3] described above, further including an active state analysis unit configured to analyze an active state on the basis of the observation signal, the body motion signal and/or the pressure signal, and determine a reference signal from the body motion signal or the pressure signal on the basis of the analysis result.
[5] The biological information processing apparatus according to any one of [1] to [4] described above, further including a bandpass filter unit configured to extract a fluctuation component from the signal using a bandpass filter.
[6] The biological information processing apparatus according to any one of [1] to [5] described above, further including an output signal quality calculation unit configured to determine a reduction state of body motion noise on the basis of a relationship between observation signal power calculated from the observation signal and error signal power calculated from the error signal.
[7] The biological information processing apparatus according to any one of [1] to [6] described above, further including a postprocessing filter unit configured to further reduce residual noise included in the error signal, by low pass filter processing.
[8] The biological information processing apparatus according to any one of [1] to [7] described above,

in which the active state analysis unit further includes a second sensor analysis unit configured to determine an active state, and

the active state analysis unit is configured to output, in a case where it is determined in the second sensor analysis unit that the body motion signal is equal to or larger than a threshold value, the body motion signal to the noise reduction processing unit as a reference signal.

[9] The biological information processing apparatus according to any one of [1] to [8] described above,

in which the active state analysis unit further includes a third sensor analysis unit configured to determine a quasi-rest state, and

the active state analysis unit is configured to output, in a case where it is determined in the third sensor analysis unit that the pressing force signal is equal to or larger than a threshold value, the pressing force signal to the noise reduction processing unit as a reference signal.

[10] The biological information processing apparatus according to any one of [1] to [9] described above, in which the active state analysis unit is configured to output, in a case where it is determined in the third sensor analysis unit that the pressure signal is smaller than a threshold value, the observation signal as-is to the noise reduction processing unit.
[11] The biological information processing apparatus according to any one of [1] to [10] described above,

in which the active state analysis unit further includes a first sensor analysis unit configured to determine non-attachment or noncontact, and

the first sensor analysis unit is configured to determine, in a case where it is determined in the first sensor analysis unit that the observation signal is smaller than a threshold value, non-attachment or noncontact.

[12] The biological information processing apparatus according to any one of [1] to [11] described above, further including a preprocessing unit configured to perform absolute value processing of a signal on a fluctuation component subjected to bandpass filter processing, as preprocessing of a signal to be input to the noise reduction processing unit.
[13] The biological information processing apparatus according to any one of [1] to [12] described above,

in which the noise reduction processing unit further includes an adaptive filter processing unit, and

the noise reduction processing unit is configured to calculate an error signal obtained by subtracting a reference signal of the adaptive filter processing unit as body motion noise from an observation signal.

[14] The biological information processing apparatus according to any one of [1] to [13] described above, in which the adaptive filter processing unit is configured to add a transfer function of a band that is calculated from a pressing force signal difference between a pressing force change in skin, and a pressing force change between band materials, to a fluctuation component of a pressing force change that has been subjected to bandpass filter processing, and use as a reference signal.
[15] The biological information processing apparatus according to any one of [1] to [14] described above, in which the biological information processing apparatus is a band type.
[16] A noise reduction processing method in biological information processing, the noise reduction processing method including:

calculating an error signal obtained by subtracting body motion noise included in an observation signal from a first sensor configured to measure biological body affect as an observation signal,

on the basis of a body motion signal from a second sensor configured to measure a body motion change, and/or a pressure signal from a third sensor configured to measure a pressing force change between a skin.

[17] The noise reduction processing method according to [16] described above, further including performing active state analysis in an order of the observation signal, the body motion signal, and/or the pressure signal, and determining body motion noise on the basis of the analysis result.

### REFERENCE SIGNS LIST

61 Contact analysis unit
62 Body motion analysis unit
63 Pressing force analysis unit
100 Biological information processing apparatus
140 Biological sensor module
141 Wristband
150 Sensor unit
151 First sensor
152 Second sensor
153 Third sensor
160 Processing unit
161 Noise reduction processing unit
162 Active state analysis unit
166 Adaptive filter processing unit
200 Network
300 Server

1. A biological information processing apparatus comprising:
a noise reduction processing unit configured to calculate an error signal obtained by subtracting body motion noise included in an observation signal from a first sensor unit configured to measure biological body affect as an observation signal,
on a basis of a body motion signal from a second sensor unit configured to measure a body motion change, and/or a pressure signal from a third sensor unit configured to measure a pressing force change in skin.

2. The biological information processing apparatus according to claim 1, wherein the first sensor is a sweat sensor unit.

3. The biological processing information apparatus according to claim 1, wherein the noise reduction processing unit is configured to regard either the body motion signal or the pressure signal as a reference signal, and calculate an error signal by subtracting body motion noise from the observation signal, using the reference signal.

4. The biological information processing apparatus according to claim 3, further comprising an active state analysis unit configured to analyze an active state on a basis of the observation signal, the body motion signal and/or the pressure signal, and determine a reference signal from the body motion signal or the pressure signal on a basis of the analysis result.

5. The biological information processing apparatus according to claim 1, further comprising a bandpass filter unit configured to extract a fluctuation component from the signal using a bandpass filter.

6. The biological information processing apparatus according to claim 1, further comprising an output signal quality calculation unit configured to determine a reduction state of body motion noise on a basis of a relationship between observation signal power calculated from the observation signal and error signal power calculated from the error signal.

7. The biological information processing apparatus according to claim 1, further comprising a postprocessing filter unit configured to further reduce residual noise included in the error signal, by low pass filter processing.

8. The biological information processing apparatus according to claim 3,
wherein the active state analysis unit further includes a second sensor analysis unit configured to determine an active state, and
the active state analysis unit is configured to output, in a case where it is determined in the second sensor analysis unit that the body motion signal is equal to or larger than a threshold value, the body motion signal to the noise reduction processing unit as a reference signal.

9. The biological information processing apparatus according to claim 3,
wherein the active state analysis unit further includes a third sensor analysis unit configured to determine a quasi-rest state, and
the active state analysis unit is configured to output, in a case where it is determined in the third sensor analysis unit that the pressing force signal is equal to or larger than a threshold value, the pressing force signal to the noise reduction processing unit as a reference signal.

10. The biological information processing apparatus according to claim 7, wherein the active state analysis unit is configured to output, in a case where it is determined in the third sensor analysis unit that the pressure signal is smaller than a threshold value, the observation signal as-is to the noise reduction processing unit.

11. The biological information processing apparatus according to claim 3,
wherein the active state analysis unit further includes a first sensor analysis unit configured to determine non-attachment or noncontact, and
the first sensor analysis unit is configured to determine, in a case where it is determined in the first sensor analysis unit that the observation signal is smaller than a threshold value, non-attachment or noncontact.

12. The biological information processing apparatus according to claim 1, further comprising a preprocessing unit configured to perform absolute value processing of a signal on a fluctuation component subjected to bandpass filter processing, as preprocessing of a signal to be input to the noise reduction processing unit.

13. The biological information processing apparatus according to claim 1,
wherein the noise reduction processing unit further includes an adaptive filter processing unit, and
the noise reduction processing unit is configured to calculate an error signal obtained by subtracting a reference signal of the adaptive filter processing unit as body motion noise from an observation signal.

14. The biological information processing apparatus according to claim 13, wherein the adaptive filter processing unit is configured to add a transfer function of a band that is calculated from a pressing force signal difference between a pressing force change in skin, and a pressing force change between band materials, to a fluctuation component of a pressing force change that has been subjected to bandpass filter processing, and use as a reference signal.

15. A noise reduction processing method in biological information processing, the noise reduction processing method comprising:
calculating an error signal obtained by subtracting body motion noise included in an observation signal from a first sensor configured to measure biological body affect as an observation signal,
on a basis of a body motion signal from a second sensor configured to measure a body motion change, and/or a pressure signal from a third sensor configured to measure a pressing force change in skin.
